(19) United States
(12) Patent Application Publication
(10) Pub. No.: US 2010/0324133 A1
(43) Pub. Date: Dec. 23, 2010

(54) Branched Diepoxide Compounds for the Treatment of Inflammatory Disorders

(76) Inventor: Dennis C. Liotta, Atlanta, GA (US)

Correspondence Address: KING & SPALDING, 1180 PEACHTREE STREET , NE, ATLANTA, GA, 30309-3521, US

(21) Appl. No.: 12/831,672

(22) Filed: Jul. 7, 2010

Related U.S. Application Data
(63) Continuation of application No. PCT/US09/30338, filed on Jan. 7, 2009.
(60) Provisional application No. 61/019,559, filed on Jan. 7, 2008.
(60) Provisional application No. 61/019,650, filed on Jan. 8, 2008.

(51) Int. Cl. A61K 31/336; A61P 35/00; C07D 303/12

(52) U.S. Cl. 514/475; 549/332

(57) ABSTRACT

The present invention provides certain diepoxide carbocyclic compounds wherein at least one carbocyclic ring carbon includes two non-epoxide substituents, and pharmaceutical compositions containing the same, for the treatment or prophylaxis of inflammatory, autoimmune and hyper- or abnormally proliferative diseases and disorders.

### CROSS REFERENCE TO RELATED APPLICATIONS

The present application claims the benefit of U.S. Provisional Patent Application Nos. 61/019,559, filed Jan. 7, 2008 and 61/019,650, filed Jan. 8, 2008, both of which are incorporated herein by reference in their entirety.

### FIELD OF THE INVENTION

The present invention is in the area of pharmaceutical chemistry and specifically relates to certain branched diepoxide compounds and pharmaceutical compositions for the treatment of inflammatory diseases as well as disorders characterized by abnormal cell proliferation.

### BACKGROUND OF THE INVENTION

Autoimmune and inflammatory diseases affect more than fifty million Americans. The immune system functions as the body's major defense against diseases caused by invading organisms. This complex system fights disease by killing invaders such as bacteria, viruses, parasites or cancerous cells while leaving the body's normal tissues unharmed. The immune system's ability to distinguish the body's normal tissues, or self, from foreign or cancerous tissue, or non-self, is an essential feature of normal immune system function. A second essential feature is memory, the ability to remember a particular foreign invader and to mount an enhanced defensive response when the previously encountered invader returns. The loss of recognition of a particular tissue as self and the subsequent immune response directed against that tissue produce serious illness.

As a result of basic research in molecular and cellular immunology over the last ten to fifteen years, approaches to diagnosing, treating and preventing these immunological based diseases have been changed. By dissecting the individual components of the immune system, those cells, receptors and mediators that are critical to the initiation and progression of immune responses have been, and continue to be, elucidated. Crystallographic analysis of proteins encoded in the major histocompatability complex, identification of an antigen-specific T cell receptor, and development of a basic understanding of the complex cytokine network have all contributed to a revolution in immunology. Various immunosuppressive agents have proved to be somewhat useful in the prevention of transplantation rejection and in the treatment of autoimmune diseases such as rheumatoid arthritis, nephritis, uveitis, thyroiditis, and early stage of insulin dependent diabetes mellitus, systemic lupus erythematosus, psoriasis and inflammatory bowel disease. However, none of the available drugs are completely effective and most are limited by severe toxicity.

In multicellular organisms, homeostasis is supported by a carefully regulated balance between cell proliferation and cell death (apoptosis). Cell proliferation is a highly regulated process involving positive regulation by growth factors and proto-oncogenes as well as negative regulation by tumor suppressor genes (Rozengurt E. “Growth factors and cell proliferation” Curr. Opin. Cell Biol. 1992 4:161; Levine A J. “The tumor suppressor genes” Annu. Rev. Biochem. 1993 62:623). A number of diverse human pathologies are known to be associated with uncontrolled cell proliferation, including cancer. Cancer is a disease with profound personal and economic costs. A huge number of people are either directly or indirectly affected by the disease. The National Cancer Institute estimates that approximately 8.9 million Americans with a history of cancer were alive in 1997. Some of these individuals were considered cured, while others still had evidence of the disease. The economic costs of cancer are also great. The National Institutes of Health estimate overall costs for cancer in the year 2001 at $1.7 billion.

Abnormal proliferation of vascular smooth muscle cells has been implicated in forms of human pathogenesis other than cancer. Benign tumors are characterized by abnormal cell proliferation but are not malignant, recurrent, invasive or progressive. Yet, due to metabolic effects or critical location (e.g., the brain), certain benign tumors can have devastating consequences.

Many disorders of abnormal cell proliferation also implicate an overstimmulated immune response. For example, rheumatoid arthritis (RA) is a chronic inflammatory disease characterized by hyperplasia of the synovial lining cells which destroy cartilage in the RA joint (Harris E D J. N Engl J Med 1990 322:1277-89; Volin M V and Koch A E. Frontiers in Bioscience 2000 5:D594-601). The altered rates of proliferation and apoptosis of RA synovial cells result in the hyperplasia of synovial tissue and in concert with the chronic inflammatory environment ultimately lead to the destruction of the RA joint. Despite the investment of billions of dollars, cures and treatments for hyperpoliferative disorders and chronic pathological inflammation remain illusive. Treatments for abnormal cell proliferative disorders generally include chemotherapy, radiation therapy, immunotherapy and gene therapy while chronic inflammatory disorders are treated with a variety of anti-inflammatory agents as discussed above. Although each treatment approach is associated with particular advantages and disadvantages, there remains a significant clinical need for novel therapeutics for the treatment of abnormal cell proliferation and chronic inflammatory disorders.

Triptolide

It has now been discovered that various extracts from the poisonous plant Tripterygium wilfordii play an important part in autoimmune and inflammatory suppression as well as cell proliferation. A number of compounds having immuno-suppressive or other activities have been isolated from extracts of root tissues from T. wilfordii, including tripterinin (PCT Application PCT/US94/02540), 16-hydroxytriptolide (Ma, 1991a; 1992a), triptriolide (Ma, 1991b), celastrol (Zhang, 1986a,b), tripchlorolide (Zhang, 1992), triptophenolide (Deng, 1992), triptonide (Wu, 1992), tripterine (Zhang, 1990a), tripterygic acid (Zhang, 1990b), sesquiterpene alkaloids (Ya, 1990), isowilfordine (Ya, 1991), sesquiterpene esters (Takaishi, 1990; 1991a; 1992a), sesquiterpene polyol esters (Takaishi, 1991b,c), phenanthrene derivatives (Takaishi, 1991d) tripterygone (Zhang, 1991), salaspermic acid (Chen, 1992), other diterpene lactone epoxide compounds (Zheng, 1991; Ma, 1992b), and diterpene quinones (Shen, 1992; Takaishi, 1992b; Shishido, 1993). The compound triptolide, which contains and unusual triepoxide moiety and an α,β unsaturated γ-lactone in the diterpene skeleton, has also been isolated from T. wilfordii and has been found to have potent anti-proliferative properties.

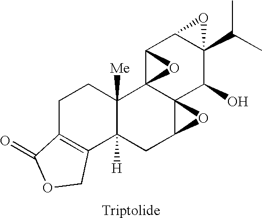

At present, it is not known whether cultured cells from T. wilfordii could be induced to produce any of these compounds in commercially useful amounts.

U.S. Pat. No. 6,777,441 and WO 2002/028862 both to Venkatesan et al. describe certain analogs of triptolide useful for the treatment of autoimmune and inflammatory diseases. Also described are pharmaceutical compositions comprising the triptolide analogs and methods of treatment of autoimmune and inflammatory diseases with the compounds.

There remains a need for additional improved anti-proliferative and anti-inflammatory or immunosuppressive compounds. In particular, there remains a need for improved anti-proliferative and anti-inflammatory or immunosuppressive compounds having improved water solubility and low toxicity.

### SUMMARY OF THE INVENTION

Certain compounds, pharmaceutical compositions and methods for the treatment or prophylaxis of autoimmune or inflammatory disorders, especially chronic inflammatory disorders, and disorders of abnormal cell proliferation, are provided; wherein the compounds include a carbocyclic ring comprising two epoxide groups, wherein at least one non-epoxide linked carbon of the carbocyclic ring carries two non-hydrogen substituents.

In particular, compounds, pharmaceutical compositions including the compounds and a pharmaceutically acceptable carrier, as well as methods of treatment or prophylaxis of inflammatory, autoimmune or proliferative disorders including administering a compound to a host in need thereof are provided, wherein the compounds are of the formulae (I)-(VI):

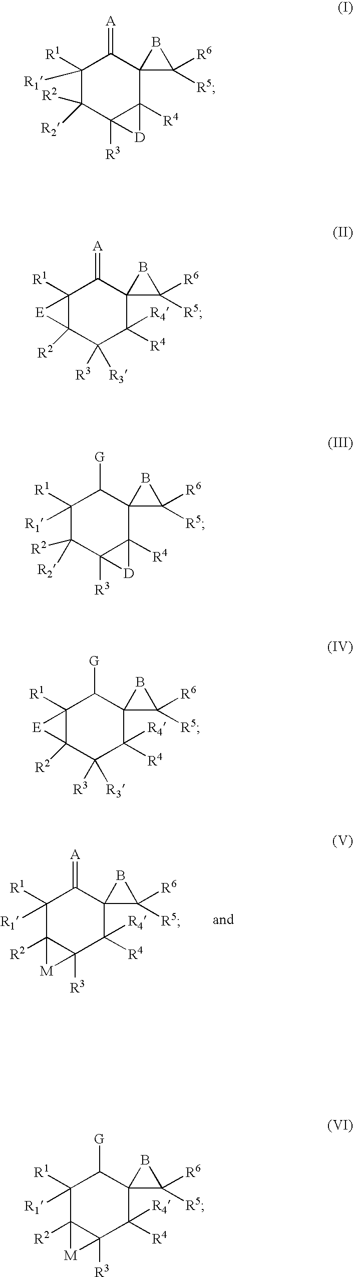

and their pharmaceutically acceptable salts, esters or prodrugs, wherein:

each A, B, D, E and M is independently O, S, NR⁷ or CR⁷R⁸;

each G is OR¹¹;NR¹¹R¹² or SR¹¹;

each R¹, R², R³, R⁴, R⁵ and R⁶ is independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, carbonyl, carboxylic acid, ester, carbamate, amide, amine, hydroxyl, alkoxy, nitro, cyano, azide, sulfonyl, sulfanyl, sulfinyl, sulfamoyl, phosphonyl, phosphinyl, phosphoryl, phosphine, a residue of a natural or synthetic amino acid, a residue of a natural or synthetic carbohydrate or XR⁹ (wherein X═O, S or NR¹⁰);

alternatively, one or more of R¹ or R^{1′} and R² or R^{2′}, R² or R^{2′} and R³ or R^{3′}, R³ or R^{3′} and R⁴ or R^{4′}, R⁴ or R^{4′} and R⁵, or R⁵ and R⁶, come together to form a bridged compound, preferably as a 3, 5, 6 or 7 membered ring, to form a cycloalkyl, cycloalkenyl, cycloalkynyl, aryl, heterocyclic, or heteroaryl;

R^{1′}, R^{2′}, R^{3′} and R^{4′} are independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, carbonyl, carboxylic acid, ester, carbamate, amide, amine, hydroxyl, alkoxy, nitro, cyano, azide, sulfonyl, sulfanyl, sulfinyl, sulfamoyl, phosphonyl, phosphinyl, phosphoryl, phosphine, halogen, —OH, —OR⁹, —NH₂, —NHR⁹, —N(R⁹)₂, —SH, —SR⁹, —OC(O)R⁹, —OC(O)OR⁹, —OC(O)N(R⁹)₂, —NR⁹C(O)R⁹, —NR⁹C(O)OR⁹, —NR⁹C(O)N(R⁹)₂, —NR⁹SO₂R⁹, —SO₂N(R⁹)₂, —S(O)R⁹, —S(O)₂R⁹, —N—OR⁹, a residue of a natural or synthetic amino acid, a residue of a natural or synthetic carbohydrate or XR⁹ (wherein X═O, S or NR¹⁰); and

each R⁷, R⁸, R⁹, R¹⁰, R¹¹ and R¹² is independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, a residue of a natural or synthetic amino acid or a residue of a natural or synthetic carbohydrate;

with the proviso that in compounds of Formula I or III, when R¹ or R^{1′} are hydrogen, then R² and R^{2′} are not hydrogen, and that when R² or R^{2′} are hydrogen, then R¹⁺ and R^{1′} are not hydrogen; in Formula II and IV, when R³ or R^{3′} are hydrogen, then R⁴ and R^{4′} are not hydrogen, and that when R⁴ or R^{4′} are hydrogen, then R³ and R^{3′} are not hydrogen; and in Formula V and VI, when R¹ or R^{1′} are hydrogen, then R⁴ and R^{4′} are not hydrogen, and that when R⁴ or R^{4′} are hydrogen, then R¹ and R^{1′} are not hydrogen.

In one embodiment, the compounds are of Formulae I or II. In these embodiments, typically the variables A, B, D and E are O. In another embodiment, R^{1′}, R^{2′}, R^{3′} and R^{4′} are independently —OH, —OR⁹, —NH₂, —NHR⁹, or —N(R⁹)₂.

In some embodiments the variables R¹, R², R⁴, R⁵ and R⁶ are independently hydrogen or alkyl. In another embodiment, variables R⁵ and R⁶ are hydrogen. In still another embodiment, R³ is hydrogen. In another embodiment variable R⁴ is alkyl.

In a particular embodiment a compound of formulae (I) or (II) is presented wherein: R¹ and R² are independently hydrogen or alkyl;

R⁴ is hydrogen or alkyl;

R⁵ and R⁶ are hydrogen; and

A, B, D and E are O.

In a subembodiment of the compound of formula (I) above, variables R^{1′} and R^{2′} are independently OH or NH₂. In another embodiment of formula (II), variables R^{3′} and R^{4′} are independently OH or NH₂. In some further embodiments of formulae (I) or (II), the group R¹ is methyl, cyclohexyl, isopropyl or tert-butyl.

In a particular embodiment of the invention, a compound with the formula shown below is provided.

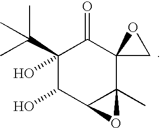

In another particular embodiment, a compound with the formula below is provided

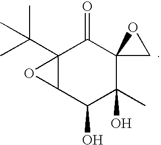

In one embodiment, the compound exhibits an EC₅₀ of less than 25, 15, 10, 5 or 1 micromolar when tested in an in vitro or in vivo inflammation or in vitro or in vivo cell proliferation assay.

In one embodiment, a method of treatment or prophylaxis of an autoimmune or inflammatory disease or disorder, particularly a chronic inflammatory disease is provided comprising administering an effective amount of an active compound of the present invention, optionally in a pharmaceutically acceptable carrier, optionally in combination or alternation with one or more other active agent. In certain embodiments, the second active agent is an immunosuppressant or anti-inflammatory agent. In certain particular embodiments, the disease or disorder is rheumatoid arthritis. In other specific embodiments, the disease or disorder is an organ transplant rejection response.

In yet another embodiment, a method for the treatment or prophylaxis of disease or disorder associated with abnormal cell proliferation in a host is provided, comprising administering an effective amount of an active compound of the present invention, optionally in a pharmaceutically acceptable carrier, optionally in combination or alternation with one or more other anti-proliferative or anti-cancer agents.

The compounds of the present invention can be administered in a pharmaceutical compositions, with a pharmaceutically acceptable carrier or diluent. In one embodiment, the active compounds of the invention can be administered in combination or alternation with one or more other active agents, including other immunosuppressant, anti-inflammatory, anti-proliferative or anti-cancer agents. In combination therapy, effective dosages of two or more agents are administered together, whereas during alternation therapy an effective dosage of each agent is administered serially. The dosages will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. In one embodiment, compositions comprising the compounds of the present invention, optionally in a pharmaceutically acceptable carrier or diluent, in combination with one or more other active agent, including another anti-proliferative or anti-cancer agent are provided.

### BRIEF DESCRIPTION OF THE FIGURES

FIG. 1 depicts the reactivity of the two cyclohexadienone bonds to epoxidizing agents.

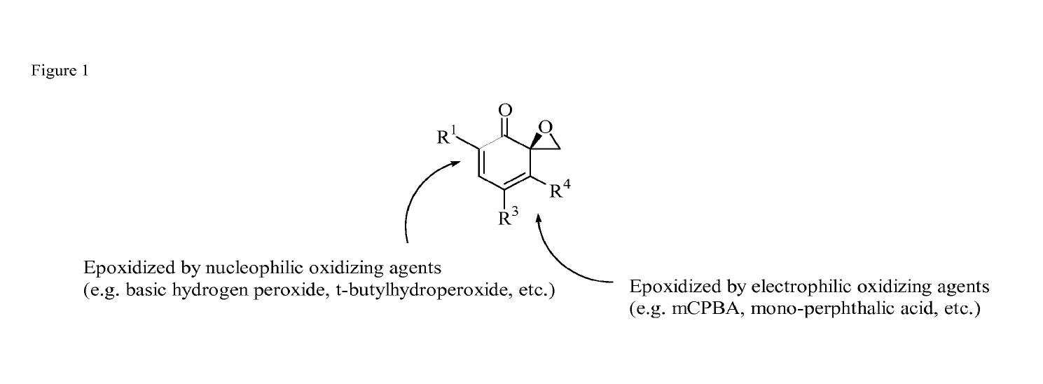

### DETAILED DESCRIPTION OF THE INVENTION

The present invention provides certain diepoxy, carboxyclic compounds that include at least one “branched” carbon with two non-epoxy substituents, pharmaceutical compositions containing these and methods for the treatment of autoimmune, inflammatory or hyper- or abnormally proliferative diseases or disorders characterized by abnormal cell proliferation.

### I. COMPOUNDS OF THE PRESENT INVENTION

### Formula I

In one embodiment of the present invention a compound of the formula (I) is provided:

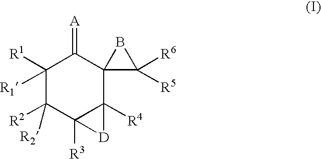

or its pharmaceutically acceptable salt, esters or prodrug, wherein:

A, B and D are independently O, S, NR⁷ or CR⁷R⁸;

R¹, R², R³, R⁴, R⁵ and R⁶ are independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, carbonyl, carboxylic acid, ester, carbamate, amide, amine, hydroxyl, alkoxy, nitro, cyano, azide, sulfonyl, sulfanyl, sulfinyl, sulfamoyl, phosphonyl, phosphinyl, phosphoryl, phosphine, a residue of a natural or synthetic amino acid, a residue of a natural or synthetic carbohydrate or XR⁹ (wherein X═O, S or NR¹⁰);

alternatively, one or more of R¹ or R^{1′} and R² or R^{2′}, R² or R^{2′} and R³, R³ and R⁴, R⁴ and R⁵, or R⁵ and R⁶, come together to form a bridged compound, preferably as a 3, 5, 6 or 7 membered ring, to form a cycloalkyl, cycloalkenyl, cycloalkynyl, aryl, heterocyclic, or heteroaryl;

each R⁷, R⁸, R⁹ and R¹⁰ is independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, a residue of a natural or synthetic amino acid or a residue of a natural or synthetic carbohydrate;

R^{1′} and R^{2′} are independently hydrogen, alkyl, aryl, heterocyclyl, heteroaryl, halogen, azide, —OH, —OR⁹, —NH₂, —NHR⁹, —N(R⁹)₂, —SH, —SR⁹, —OC(O)R⁹, —OC(O)OR⁹, —OC(O)N(R⁹)₂, —NR⁹C(O)R⁹, —NR⁹C(O)OR⁹, —NR⁹C(O)N(R⁹)₂, —NR⁹SO₂R⁹, —SO₂N(R⁹)₂, —S(O)R⁹, —S(O)₂R⁹, —N—OR⁹, a residue of a natural or synthetic amino acid, a residue of a natural or synthetic carbohydrate;

wherein two R⁹ groups on the same nitrogen may form an optionally substituted 3-8 membered heterocyclic or heteroaryl ring;

with the proviso that when R¹ or R^{1′} are hydrogen, then R² and R^{2′} are not hydrogen, and that when R² or R^{2′} are hydrogen, then R¹ and R^{1′} are not hydrogen.

In a first principle embodiment of formula (I), A is O. In one subembodiment, B is O and D is O. In another subembodiment, B is O and D is S. In still another subembodiment, B is O and D is NR⁷. In another subembodiment, B is O and D is CR⁷R⁸. In another subembodiment, B is S and D is O. In another subembodiment, B is S and D is S. In still another embodiment, B is S and D is NR⁷. In another subembodiment, B is S and D is CR⁷R⁸. In another subembodiment, B is NR⁷ and D is O. In another subembodiment, B is NR⁷ and D is S. In still another subembodiment, B is NR⁷ and D is NR⁷. In another subembodiment, B is NR⁷ and D is CR⁷R⁸. In yet another subembodiment, B is CR⁷R⁸ and D is O. In another subembodiment, B is CR⁷R⁸ and D is S. In another subembodiment, B is CR⁷R⁸ and D is NR⁷. In still another subembodiment, B is CR⁷R⁸ and D is CR⁷R⁸.

In another subembodiment, R^{1′} and R^{2′} are independently hydrogen, halogen, azide, —OH, —OR⁹, —NH₂, —NHR⁹, —N(R⁹)₂, —SH, —SR⁹, —OC(O)R⁹, —OC(O)OR⁹, —OC(O)N(R⁹)₂, a residue of a natural or synthetic amino acid, a residue of a natural or synthetic carbohydrate; wherein two R⁹ groups on the same nitrogen may form an optionally substituted 3-8 membered heterocyclic or heteroaryl ring.

In another subembodiment, R^{1′} and R^{2′} are independently hydrogen, halogen, azide, —OH, —OR⁹, —NH₂, —NHR⁹, —N(R⁹)₂, —SH, —SR⁹, —OC(O)R⁹, —OC(O)OR⁹, —NR⁹C(O)R⁹, —NR⁹C(O)OR⁹, or —NR⁹SO₂R⁹; wherein two R⁹ groups on the same nitrogen may form an optionally substituted 3-8 membered heterocyclic or heteroaryl ring.

In still another embodiment, R^{1′} and R^{2′} are independently hydrogen, —OH, —OR⁹, —NH₂, —NHR⁹, or —N(R⁹)₂; wherein two R⁹ groups on the same nitrogen may form an optionally substituted 3-8 membered heterocyclic or heteroaryl ring.

In another embodiment, R^{1′} and R^{2′} are independently hydrogen, OR⁹, N(R⁹)₂ or a residue of a natural or synthetic amino acid.

In still another embodiment, R¹, R², R³, R⁴, R⁵ and R⁶ are independently hydrogen, alkyl, alkenyl, alkynyl, cycloalkyl, cycloalkenyl, aryl, alkaryl, arylalkyl, heterocyclic, or heteroaryl.

In another subembodiment, R^{1′} and R^{2′} are independently hydrogen, —OH, —OR⁹, —NH₂, —NHR⁹, or —N(R⁹)₂; and R¹, R², R³ and R⁴ are independently hydrogen or alkyl.

In another subembodiment, R^{1′} and R^{2′} are independently —OH or —OR⁹; and R¹ and R² are independently hydrogen or alkyl.

In yet another embodiment, R¹ is methyl, t-butyl, cyclohexyl or isopropyl; R², R³, R⁵ and R⁶ are hydrogen; R⁴ is methyl; and R^{1′} and R^{2′} are hydroxy or OR⁹.

In one embodiment, R^{1′} and R^{2′} are oriented in a cis-relationship to each other. In another embodiment, R^{1′} and R^{2′} are oriented in a trans-relationship to each other.

In still another subembodiment, R¹ is methyl, isopropyl, cyclohexyl or tert-butyl, R² is hydrogen, and R⁴ is methyl.

In a particular embodiment of the invention, the compound of formula I is

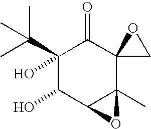

or its pharmaceutically acceptable salt, ester or prodrug.

In another particular embodiment, the compound of formula I is

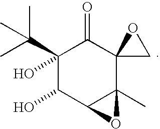

In another particular embodiment, the compound of formula I is

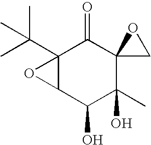

or its pharmaceutically acceptable salt, ester or prodrug.

In another particular embodiment, the compound of formula I is

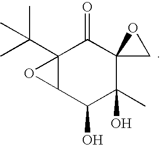

In one embodiment, the invention provides a monoepoxide intermediate used in the synthesis of the diepoxide compounds of the invention. In another embodiment, a branched monoepoxide intermediate is provided. In still another embodiment, a dihydroxylated monoepoxide intermediate is provided.

In a particular embodiment of the present invention, the compounds of the formula (I) are the following species:

(I)

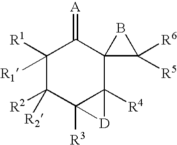

| A | B | D | R¹ | R^{1′} | R² | R^{2′} | R³ | R⁴ | R⁵ | R⁶ |
|---|---|---|---|---|---|---|---|---|---|---|
| O | O | O | Me | OH | H | OH | H | H | H | H |
| O | O | O | i-Pr | OH | H | OH | H | H | H | H |
| O | O | O | t-Bu | OH | H | OH | H | H | H | H |
| O | O | O | Ph | OH | H | OH | H | H | H | H |
| O | O | O | i-Pr | OH | Me | OH | H | H | H | H |
| O | O | O | Me | OH | Me | OH | H | H | H | H |
| O | O | O | t-Bu | OH | Me | OH | H | H | H | H |
| O | O | O | Ph | OH | Me | OH | H | H | H | H |
| O | O | O | Me | OH | H | OH | Me | H | H | H |
| O | O | O | i-Pr | OH | H | OH | Me | H | H | H |
| O | O | O | t-Bu | OH | H | OH | Me | H | H | H |
| O | O | O | Ph | OH | H | OH | Me | H | H | H |
| O | O | O | Me | OH | H | OH | H | Me | H | H |
| O | O | O | i-Pr | OH | H | OH | H | Me | H | H |
| O | O | O | t-Bu | OH | H | OH | H | Me | H | H |
| O | O | O | Ph | OH | H | OH | H | Me | H | H |
| O | O | O | Me | OH | H | OH | CH₂Ph | H | H | H |
| O | O | O | i-Pr | OH | H | OH | CH₂Ph | H | H | H |
| O | O | O | t-Bu | OH | H | OH | CH₂Ph | H | H | H |
| O | O | O | Ph | OH | H | OH | CH₂Ph | H | H | H |
| O | O | O | Me | OH | H | H | H | H | H | H |
| O | O | O | i-Pr | OH | H | H | H | H | H | H |
| O | O | O | Ph | OH | H | H | H | H | H | H |
| O | O | O | Me | OH | Me | H | H | H | H | H |
| O | O | O | i-Pr | OH | Me | H | H | H | H | H |
| O | O | O | t-Bu | OH | Me | H | H | H | H | H |
| O | O | O | Ph | OH | Me | H | H | H | H | H |
| O | O | O | Me | OH | H | H | Me | H | H | H |
| O | O | O | i-Pr | OH | H | H | Me | H | H | H |
| O | O | O | t-Bu | OH | H | H | Me | H | H | H |
| O | O | O | Ph | OH | H | H | Me | H | H | H |
| O | O | O | Me | OH | H | H | H | Me | H | H |
| O | O | O | i-Pr | OH | H | H | H | Me | H | H |
| O | O | O | t-Bu | OH | H | H | H | Me | H | H |
| O | O | O | Ph | OH | H | H | H | Me | H | H |
| O | O | O | Me | OH | H | H | CH₂Ph | H | H | H |
| O | O | O | i-Pr | OH | H | H | CH₂Ph | H | H | H |
| O | O | O | t-Bu | OH | H | H | CH₂Ph | H | H | H |
| O | O | O | Ph | OH | H | H | CH₂Ph | H | H | H |
| O | O | O | Me | H | Me | OH | H | H | H | H |
| O | O | O | i-Pr | H | Me | OH | H | H | H | H |
| O | O | O | t-Bu | H | Me | OH | H | H | H | H |
| O | O | O | Ph | H | Me | OH | H | H | H | H |
| O | O | O | Me | NH₂ | H | OH | H | H | H | H |
| O | O | O | i-Pr | NH₂ | H | OH | H | H | H | H |
| O | O | O | Ph | NH₂ | H | OH | H | H | H | H |
| O | O | O | Me | NH₂ | Me | OH | H | H | H | H |
| O | O | O | i-Pr | NH₂ | Me | OH | H | H | H | H |
| O | O | O | t-Bu | NH₂ | Me | OH | H | H | H | H |
| O | O | O | Ph | NH₂ | Me | OH | H | H | H | H |
| O | O | O | Me | NH₂ | H | OH | Me | H | H | H |
| O | O | O | i-Pr | NH₂ | H | OH | Me | H | H | H |
| O | O | O | t-Bu | NH₂ | H | OH | Me | H | H | H |
| O | O | O | Ph | NH₂ | H | OH | Me | H | H | H |
| O | O | O | Me | NH₂ | H | OH | H | Me | H | H |
| O | O | O | i-Pr | NH₂ | H | OH | H | Me | H | H |
| O | O | O | t-Bu | NH₂ | H | OH | H | Me | H | H |
| O | O | O | Ph | NH₂ | H | OH | H | Me | H | H |
| O | O | O | Me | NH₂ | H | OH | CH₂Ph | H | H | H |
| O | O | O | i-Pr | NH₂ | H | OH | CH₂Ph | H | H | H |
| O | O | O | t-Bu | NH₂ | H | OH | CH₂Ph | H | H | H |
| O | O | O | Ph | NH₂ | H | OH | CH₂Ph | H | H | H |
| O | O | O | Me | OH | H | NH₂ | H | H | H | H |
| O | O | O | i-Pr | OH | H | NH₂ | H | H | H | H |
| O | O | O | Ph | OH | H | NH₂ | H | H | H | H |
| O | O | O | Me | OH | Me | NH₂ | H | H | H | H |
| O | O | O | i-Pr | OH | Me | NH₂ | H | H | H | H |
| O | O | O | t-Bu | OH | Me | NH₂ | H | H | H | H |
| O | O | O | Ph | OH | Me | NH₂ | H | H | H | H |
| O | O | O | Me | OH | H | NH₂ | Me | H | H | H |
| O | O | O | i-Pr | OH | H | NH₂ | Me | H | H | H |
| O | O | O | t-Bu | OH | H | NH₂ | Me | H | H | H |
| O | O | O | Ph | OH | H | NH₂ | Me | H | H | H |
| O | O | O | Me | OH | H | NH₂ | H | Me | H | H |
| O | O | O | i-Pr | OH | H | NH₂ | H | Me | H | H |
| O | O | O | t-Bu | OH | H | NH₂ | H | Me | H | H |
| O | O | O | Ph | OH | H | NH₂ | H | Me | H | H |
| O | O | O | Me | OH | H | NH₂ | CH₂Ph | H | H | H |
| O | O | O | i-Pr | OH | H | NH₂ | CH₂Ph | H | H | H |
| O | O | O | t-Bu | OH | H | NH₂ | CH₂Ph | H | H | H |
| O | O | O | Ph | OH | H | NH₂ | CH₂Ph | H | H | H |
| O | O | O | Me | NH₂ | H | H | H | H | H | H |
| O | O | O | i-Pr | NH₂ | H | H | H | H | H | H |
| O | O | O | Ph | NH₂ | H | H | H | H | H | H |
| O | O | O | Me | NH₂ | Me | H | H | H | H | H |
| O | O | O | i-Pr | NH₂ | Me | H | H | H | H | H |
| O | O | O | t-Bu | NH₂ | Me | H | H | H | H | H |
| O | O | O | Ph | NH₂ | Me | H | H | H | H | H |
| O | O | O | Me | NH₂ | H | H | Me | H | H | H |
| O | O | O | i-Pr | NH₂ | H | H | Me | H | H | H |
| O | O | O | t-Bu | NH₂ | H | H | Me | H | H | H |
| O | O | O | Ph | NH₂ | H | H | Me | H | H | H |
| O | O | O | Me | NH₂ | H | H | H | Me | H | H |
| O | O | O | i-Pr | NH₂ | H | H | H | Me | H | H |
| O | O | O | t-Bu | NH₂ | H | H | H | Me | H | H |
| O | O | O | Ph | NH₂ | H | H | H | Me | H | H |
| O | O | O | Me | NH₂ | H | H | CH₂Ph | H | H | H |
| O | O | O | i-Pr | NH₂ | H | H | CH₂Ph | H | H | H |
| O | O | O | t-Bu | NH₂ | H | H | CH₂Ph | H | H | H |
| O | O | O | Ph | NH₂ | H | H | CH₂Ph | H | H | H |
| O | O | O | Me | NH₂ | H | NH₂ | H | H | H | H |
| O | O | O | Me | H | Me | NH₂ | H | H | H | H |
| O | O | O | i-Pr | H | Me | NH₂ | H | H | H | H |
| O | O | O | t-Bu | H | Me | NH₂ | H | H | H | H |
| O | O | O | Ph | H | Me | NH₂ | H | H | H | H |

### Formula II

In another embodiment of the present invention a structure of the formula (II) is provided:

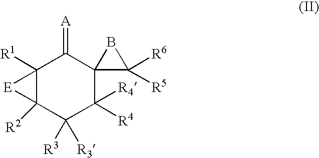

or its pharmaceutically acceptable salt, esters or prodrug, wherein:

A, B and E are independently O, S, NR⁷ or CR⁷R⁸;

R¹, R², R³, R⁴, R⁵ and R⁶ are independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, carbonyl, carboxylic acid, ester, carbamate, amide, amine, hydroxyl, alkoxy, nitro, cyano, azide, sulfonyl, sulfanyl, sulfinyl, sulfamoyl, phosphonyl, phosphinyl, phosphoryl, phosphine, a residue of a natural or synthetic amino acid, a residue of a natural or synthetic carbohydrate or XR⁹ (wherein X═O, S or NR¹⁰);

alternatively, one or more of R¹ and R², R² and R³ or R^{3′}, R³ or R^{3′} and R⁴ or R^{4′}, R⁴ or R^{4′} and R⁵, or R⁵ and R⁶, come together to form a bridged compound, preferably as a 3, 5, 6 or 7 membered ring, to form a cycloalkyl, cycloalkenyl, cycloalkynyl, aryl, heterocyclic, or heteroaryl; and

each R⁷, R⁸, R⁹ and R¹⁰ is independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, a residue of a natural or synthetic amino acid or a residue of a natural or synthetic carbohydrate; and

R^{3′} and R^{4′} are independently hydrogen, alkyl, aryl, heterocyclyl, heteroaryl, halogen, azide, —OH, —OR⁹, —NH₂, —NHR⁹, —N(R⁹)₂, —SH, —SR⁹, —OC(O)R⁹, —OC(O)OR⁹, —OC(O)N(R⁹)₂, —NR⁹C(O)R⁹, —NR⁹C(O)OR⁹, —NR⁹C(O)N(R⁹)₂, —NR⁹SO₂R⁹, —SO₂N(R⁹)₂, —S(O)R⁹, —S(O)₂R⁹, —N—OR⁹, a residue of a natural or synthetic amino acid, or a residue of a natural or synthetic carbohydrate;

wherein two R⁹ groups on the same nitrogen may form an optionally substituted 3-8 membered heterocyclic or heteroaryl ring;

with the proviso that when R³ or R^{3′} are hydrogen, then R⁴ and R^{4′} are not hydrogen, and that when R⁴ or R^{4′} are hydrogen, then R³ and R^{3′} are not hydrogen.

In a first principle embodiment of formula (II), A is O. In one subembodiment, B is O and E is O. In another subembodiment, B is O and E is S. In still another subembodiment, B is O and E is NR⁷. In another subembodiment, B is O and E is CR⁷R⁸. In another subembodiment, B is S and E is O. In another subembodiment, B is S and E is S. In still another embodiment, B is S and E is NR⁷. In another subembodiment, B is S and E is CR⁷R⁸. In another subembodiment, B is NR⁷ and E is O. In another subembodiment, B is NR⁷ and E is S. In still another subembodiment, B is NR⁷ and E is NR⁷. In another subembodiment, B is NR⁷ and E is CR⁷R⁸. In yet another subembodiment, B is CR⁷R⁸ and E is O. In another subembodiment, B is CR⁷R⁸ and E is S. In another subembodiment, B is CR⁷R⁸ and E is NR⁷. In still another subembodiment, B is CR⁷R⁸ and E is CR⁷R⁸.

In another subembodiment, R^{3′} and R^{4′} are independently hydrogen, halogen, azide, —OH, —OR⁹, —NH₂, —NHR⁹, —N(R⁹)₂, —SH, —SR⁹, —OC(O)R⁹, —OC(O)OR⁹, —OC(O)N(R⁹)₂, a residue of a natural or synthetic amino acid, or a residue of a natural or synthetic carbohydrate; wherein two R⁹ groups on the same nitrogen may form an optionally substituted 3-8 membered heterocyclic or heteroaryl ring.

In another subembodiment, R^{3′} and R^{4′} are independently hydrogen, halogen, azide, —OH, —OR⁹, —NH₂, —NHR⁹, —N(R⁹)₂, —SH, —SR⁹, —OC(O)R⁹, —OC(O)OR⁹, —NR⁹C(O)R⁹, —NR⁹C(O)OR⁹, —NR⁹C(O)N(R⁹)₂, —NR⁹SO₂R⁹,; wherein two R⁹ groups on the same nitrogen may form an optionally substituted 3-8 membered heterocyclic or heteroaryl ring.

In still another embodiment, R^{3′} and R^{4′} are independently hydrogen, —OH, —OR⁹, —NH₂, —NHR⁹, or —N(R⁹)₂; wherein two R⁹ groups on the same nitrogen may form an optionally substituted 3-8 membered heterocyclic or heteroaryl ring.

In still another embodiment, R¹, R², R³, R⁴, R⁵ and R⁶ are independently hydrogen, alkyl, alkenyl, alkynyl, cycloalkyl, cycloalkenyl, aryl, alkaryl, arylalkyl, heterocyclic, or heteroaryl.

In another subembodiment, R^{3′} and R^{4′} are independently hydrogen, —OH, —OR⁹, —NH₂, —NHR⁹, or —N(R⁹)₂; and R¹, R², R³ and R⁴ are independently hydrogen or alkyl.

In another subembodiment, R^{3′} and R^{4′} are independently —OH or —OR⁹; and R³ and R⁴ are independently hydrogen or alkyl.

In one embodiment, R^{3′} and R^{4′} are oriented in a cis-relationship to each other. In another embodiment, R^{3′} and R^{4′} are oriented in a trans-relationship to each other.
In still another subembodiment, R¹ and R⁴ are independently methyl, isopropyl, cyclohexyl or tert-butyl.

In a particular embodiment of the present invention, the compounds of the formula (II) are the following species:

(II)

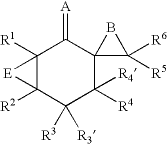

| A | B | D | R¹ | R^{3′} | R² | R^{4′} | R³ | R⁴ | R⁵ | R⁶ |
|---|---|---|---|---|---|---|---|---|---|---|
| O | O | O | Me | OH | H | OH | Me | H | H | H |
| O | O | O | i-Pr | OH | H | OH | Me | H | H | H |
| O | O | O | t-Bu | OH | H | OH | Me | H | H | H |
| O | O | O | Ph | OH | H | OH | Me | H | H | H |
| O | O | O | Me | OH | H | OH | H | Me | H | H |
| O | O | O | i-Pr | OH | H | OH | H | Me | H | H |
| O | O | O | t-Bu | OH | H | OH | H | Me | H | H |
| O | O | O | Ph | OH | H | OH | H | Me | H | H |
| O | O | O | Me | OH | H | OH | CH₂Ph | H | H | H |
| O | O | O | i-Pr | OH | H | OH | CH₂Ph | H | H | H |
| O | O | O | t-Bu | OH | H | OH | CH₂Ph | H | H | H |
| O | O | O | Ph | OH | H | OH | CH₂Ph | H | H | H |
| O | O | O | Me | OH | H | H | Me | H | H | H |
| O | O | O | i-Pr | OH | H | H | Me | H | H | H |
| O | O | O | t-Bu | OH | H | H | Me | H | H | H |
| O | O | O | Ph | OH | H | H | Me | H | H | H |
| O | O | O | Me | OH | H | H | CH₂Ph | H | H | H |
| O | O | O | i-Pr | OH | H | H | CH₂Ph | H | H | H |
| O | O | O | t-Bu | OH | H | H | CH₂Ph | H | H | H |
| O | O | O | Ph | OH | H | H | CH₂Ph | H | H | H |
| O | O | O | Me | H | H | OH | H | Me | H | H |
| O | O | O | i-Pr | H | H | OH | H | Me | H | H |
| O | O | O | t-Bu | H | H | OH | H | Me | H | H |
| O | O | O | Ph | H | H | OH | H | Me | H | H |
| O | O | O | Me | NH₂ | H | OH | Me | H | H | H |
| O | O | O | i-Pr | NH₂ | H | OH | Me | H | H | H |
| O | O | O | t-Bu | NH₂ | H | OH | Me | H | H | H |
| O | O | O | Ph | NH₂ | H | OH | Me | H | H | H |
| O | O | O | Me | NH₂ | H | OH | H | Me | H | H |
| O | O | O | i-Pr | NH₂ | H | OH | H | Me | H | H |
| O | O | O | t-Bu | NH₂ | H | OH | H | Me | H | H |
| O | O | O | Ph | NH₂ | H | OH | H | Me | H | H |
| O | O | O | Me | NH₂ | H | OH | CH₂Ph | H | H | H |
| O | O | O | i-Pr | NH₂ | H | OH | CH₂Ph | H | H | H |
| O | O | O | t-Bu | NH₂ | H | OH | CH₂Ph | H | H | H |
| O | O | O | Ph | NH₂ | H | OH | CH₂Ph | H | H | H |
| O | O | O | Me | OH | H | NH₂ | Me | H | H | H |
| O | O | O | i-Pr | OH | H | NH₂ | Me | H | H | H |
| O | O | O | t-Bu | OH | H | NH₂ | Me | H | H | H |
| O | O | O | Ph | OH | H | NH₂ | Me | H | H | H |
| O | O | O | Me | OH | H | NH₂ | H | Me | H | H |
| O | O | O | i-Pr | OH | H | NH₂ | H | Me | H | H |
| O | O | O | t-Bu | OH | H | NH₂ | H | Me | H | H |
| O | O | O | Ph | OH | H | NH₂ | H | Me | H | H |
| O | O | O | Me | OH | H | NH₂ | CH₂Ph | H | H | H |
| O | O | O | i-Pr | OH | H | NH₂ | CH₂Ph | H | H | H |
| O | O | O | t-Bu | OH | H | NH₂ | CH₂Ph | H | H | H |
| O | O | O | Ph | OH | H | NH₂ | CH₂Ph | H | H | H |
| O | O | O | Me | NH₂ | H | H | Me | H | H | H |
| O | O | O | i-Pr | NH₂ | H | H | Me | H | H | H |
| O | O | O | t-Bu | NH₂ | H | H | Me | H | H | H |
| O | O | O | Ph | NH₂ | H | H | Me | H | H | H |
| O | O | O | Me | NH₂ | H | H | CH₂Ph | H | H | H |
| O | O | O | i-Pr | NH₂ | H | H | CH₂Ph | H | H | H |
| O | O | O | t-Bu | NH₂ | H | H | CH₂Ph | H | H | H |
| O | O | O | Ph | NH₂ | H | H | CH₂Ph | H | H | H |
| O | O | O | Me | H | H | NH₂ | H | Me | H | H |
| O | O | O | i-Pr | H | H | NH₂ | H | Me | H | H |
| O | O | O | t-Bu | H | H | NH₂ | H | Me | H | H |
| O | O | O | Ph | H | H | NH₂ | H | Me | H | H |

### Formula III

In another embodiment of the present invention a structure of the formula (III) is provided:

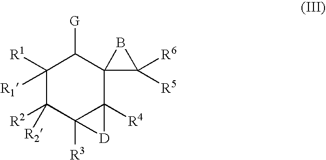

or its pharmaceutically acceptable salt, esters or prodrug, wherein:

B and D are independently O, S, NR⁷ or CR⁷R⁸;

G is OR¹¹, NR¹¹R¹² or SR¹¹;

R¹, R², R³, R⁴, R⁵ and R⁶ are independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, carbonyl, carboxylic acid, ester, carbamate, amide, amine, hydroxyl, alkoxy, nitro, cyano, azide, sulfonyl, sulfanyl, sulfinyl, sulfamoyl, phosphonyl, phosphinyl, phosphoryl, phosphine, a residue of a natural or synthetic amino acid, a residue of a natural or synthetic carbohydrate or XR⁹ (wherein X═O, S or NR¹⁰);

alternatively, one or more of R¹ or R^{1′} and R² or R^{2′}, R² or R^{2′} and R³, R³ and R⁴, R⁴ and R⁵, or R⁵ and R⁶, come together to form a bridged compound, preferably as a 3, 5, 6 or 7 membered ring, to form a cycloalkyl, cycloalkenyl, cycloalkynyl, aryl, heterocyclic, or heteroaryl; and

each R⁷, R⁸, R⁹, R¹⁰, R¹¹ and R¹² is independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, a residue of a natural or synthetic amino acid or a residue of a natural or synthetic carbohydrate; and

R^{1′} and R^{2′} are independently hydrogen, alkyl, aryl, heterocyclyl, heteroaryl, halogen, azide, —OH, —OR⁹, —NH₂, —NHR⁹, —N(R⁹)₂, —SH, —SR⁹, —OC(O)R⁹, —OC(O)OR⁹, —OC(O)N(R⁹)₂, —NR⁹C(O)R⁹, —NR⁹C(O)OR⁹, —NR⁹C(O)N(R⁹)₂, —NR⁹SO₂R⁹, —SO₂N(R⁹)₂, —S(O)R⁹, —S(O)₂R⁹, —N—OR⁹, a residue of a natural or synthetic amino acid, a residue of a natural or synthetic carbohydrate;

wherein two R⁹ groups on the same nitrogen may form an optionally substituted 3-8 membered heterocyclic or heteroaryl ring;

with the proviso that when R¹ or R^{1′} are hydrogen, then R² and R^{2′} are not hydrogen, and that when R² or R^{2′} are hydrogen, then R¹ and R^{1′} are not hydrogen.

In a first principle embodiment of formula (III), G is OR¹¹. In one subembodiment, B is O and D is O. In another subembodiment, B is O and D is S. In still another subembodiment, B is O and D is NR⁷. In another subembodiment, B is O and D is CR⁷R⁸. In another subembodiment, B is S and D is O. In another subembodiment, B is S and D is S. In still another embodiment, B is S and D is NR⁷. In another subembodiment, B is S and D is CR⁷R⁸. In another subembodiment, B is NR⁷ and D is O. In another subembodiment, B is NR⁷ and D is S. In still another subembodiment, B is NR⁷ and D is NR⁷. In another subembodiment, B is NR⁷ and D is CR⁷R⁸. In yet another subembodiment, B is CR⁷R⁸ and D is O. In another subembodiment, B is CR⁷R⁸ and D is S. In another subembodiment, B is CR⁷R⁸ and D is NR⁷. In still another subembodiment, B is CR⁷R⁸ and D is CR⁷R⁸.

In another subembodiment, R^{1′} and R^{2′} are independently hydrogen, halogen, azide, —OH, —OR⁹, —NH₂, —NHR⁹, —N(R⁹)₂, —SH, —SR⁹, —OC(O)R⁹, —OC(O)OR⁹, —OC(O)N(R⁹)₂, a residue of a natural or synthetic amino acid, a residue of a natural or synthetic carbohydrate; wherein two R⁹ groups on the same nitrogen may form an optionally substituted 3-8 membered heterocyclic or heteroaryl ring.

In another subembodiment, R^{1′} and R^{2′} are independently hydrogen, halogen, azide, —OH, —OR⁹, —NH₂, —NHR⁹, —N(R⁹)₂, —SH, —SR⁹, —OC(O)R⁹, —OC(O)OR⁹, —NR⁹C(O)R⁹, —NR⁹C(O)OR⁹, —NR⁹SO₂R⁹,; wherein two R⁹ groups on the same nitrogen may form an optionally substituted 3-8 membered heterocyclic or heteroaryl ring.

In still another embodiment, R^{1′} and R^{2′} are independently hydrogen, —OH, —OR⁹, —NH₂, —NHR⁹, or —N(R⁹)₂; wherein two R⁹ groups on the same nitrogen may form an optionally substituted 3-8 membered heterocyclic or heteroaryl ring.

In still another embodiment, R¹, R², R³, R⁴, R⁵ and R⁶ are independently hydrogen, alkyl, alkenyl, alkynyl, cycloalkyl, cycloalkenyl, aryl, alkaryl, arylalkyl, heterocyclic, or heteroaryl.

In another subembodiment, R^{1′} and R^{2′} are independently hydrogen, —OH, —OR⁹, —NH₂, —NHR⁹, or —N(R⁹)₂; and R¹, R², R³ and R⁴ are independently hydrogen or alkyl.

In another subembodiment, R^{1′} and R^{2′} are independently —OH or —OR⁹; and R¹ and R² are independently hydrogen or alkyl.

In one embodiment, R^{1′} and R^{2′} are oriented in a cis-relationship to each other. In another embodiment, R^{1′} and R^{2′} are oriented in a trans-relationship to each other.

In still another subembodiment, R¹ is methyl, cyclohexyl, isopropyl or tert-butyl; R² is hydrogen; and R⁴ is methyl.

In yet another embodiment, R¹ is methyl, t-butyl, cyclohexyl or isopropyl; R², R³, R⁵ and R⁶ are hydrogen; R⁴ is methyl; and R^{1′} and R^{2′} are hydroxy or OR⁹.

In a particular embodiment of the present invention, the compounds of the formula (III) are the following species:

(III)

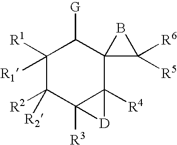

| A | B | D | R¹ | R^{1′} | R² | R^{2′} | R³ | R⁴ | R⁵ | R⁶ |
|---|---|---|---|---|---|---|---|---|---|---|
| O | O | O | Me | OH | H | OH | H | H | H | H |
| O | O | O | i-Pr | OH | H | OH | H | H | H | H |
| O | O | O | t-Bu | OH | H | OH | H | H | H | H |
| O | O | O | Ph | OH | H | OH | H | H | H | H |
| O | O | O | i-Pr | OH | Me | OH | H | H | H | H |
| O | O | O | Me | OH | Me | OH | H | H | H | H |
| O | O | O | t-Bu | OH | Me | OH | H | H | H | H |
| O | O | O | Ph | OH | Me | OH | H | H | H | H |
| O | O | O | Me | OH | H | OH | Me | H | H | H |
| O | O | O | i-Pr | OH | H | OH | Me | H | H | H |
| O | O | O | t-Bu | OH | H | OH | Me | H | H | H |
| O | O | O | Ph | OH | H | OH | Me | H | H | H |
| O | O | O | Me | OH | H | OH | H | Me | H | H |
| O | O | O | i-Pr | OH | H | OH | H | Me | H | H |
| O | O | O | t-Bu | OH | H | OH | H | Me | H | H |
| O | O | O | Ph | OH | H | OH | H | Me | H | H |
| O | O | O | Me | OH | H | OH | CH₂Ph | H | H | H |
| O | O | O | i-Pr | OH | H | OH | CH₂Ph | H | H | H |
| O | O | O | t-Bu | OH | H | OH | CH₂Ph | H | H | H |
| O | O | O | Ph | OH | H | OH | CH₂Ph | H | H | H |
| O | O | O | Me | OH | H | H | H | H | H | H |
| O | O | O | i-Pr | OH | H | H | H | H | H | H |
| O | O | O | Ph | OH | H | H | H | H | H | H |
| O | O | O | Me | OH | Me | H | H | H | H | H |
| O | O | O | i-Pr | OH | Me | H | H | H | H | H |
| O | O | O | t-Bu | OH | Me | H | H | H | H | H |
| O | O | O | Ph | OH | Me | H | H | H | H | H |
| O | O | O | Me | OH | H | H | Me | H | H | H |
| O | O | O | i-Pr | OH | H | H | Me | H | H | H |
| O | O | O | t-Bu | OH | H | H | Me | H | H | H |
| O | O | O | Ph | OH | H | H | Me | H | H | H |
| O | O | O | Me | OH | H | H | H | Me | H | H |
| O | O | O | i-Pr | OH | H | H | H | Me | H | H |
| O | O | O | t-Bu | OH | H | H | H | Me | H | H |
| O | O | O | Ph | OH | H | H | H | Me | H | H |
| O | O | O | Me | OH | H | H | CH₂Ph | H | H | H |
| O | O | O | i-Pr | OH | H | H | CH₂Ph | H | H | H |
| O | O | O | t-Bu | OH | H | H | CH₂Ph | H | H | H |
| O | O | O | Ph | OH | H | H | CH₂Ph | H | H | H |
| O | O | O | Me | H | Me | OH | H | H | H | H |
| O | O | O | i-Pr | H | Me | OH | H | H | H | H |
| O | O | O | t-Bu | H | Me | OH | H | H | H | H |
| O | O | O | Ph | H | Me | OH | H | H | H | H |
| O | O | O | Me | NH₂ | H | OH | H | H | H | H |
| O | O | O | i-Pr | NH₂ | H | OH | H | H | H | H |
| O | O | O | Ph | NH₂ | H | OH | H | H | H | H |
| O | O | O | Me | NH₂ | Me | OH | H | H | H | H |
| O | O | O | i-Pr | NH₂ | Me | OH | H | H | H | H |
| O | O | O | t-Bu | NH₂ | Me | OH | H | H | H | H |
| O | O | O | Ph | NH₂ | Me | OH | H | H | H | H |
| O | O | O | Me | NH₂ | H | OH | Me | H | H | H |
| O | O | O | i-Pr | NH₂ | H | OH | Me | H | H | H |
| O | O | O | t-Bu | NH₂ | H | OH | Me | H | H | H |
| O | O | O | Ph | NH₂ | H | OH | Me | H | H | H |
| O | O | O | Me | NH₂ | H | OH | H | Me | H | H |
| O | O | O | i-Pr | NH₂ | H | OH | H | Me | H | H |
| O | O | O | t-Bu | NH₂ | H | OH | H | Me | H | H |
| O | O | O | Ph | NH₂ | H | OH | H | Me | H | H |
| O | O | O | Me | NH₂ | H | OH | CH₂Ph | H | H | H |
| O | O | O | i-Pr | NH₂ | H | OH | CH₂Ph | H | H | H |
| O | O | O | t-Bu | NH₂ | H | OH | CH₂Ph | H | H | H |
| O | O | O | Ph | NH₂ | H | OH | CH₂Ph | H | H | H |
| O | O | O | Me | OH | H | NH₂ | H | H | H | H |
| O | O | O | i-Pr | OH | H | NH₂ | H | H | H | H |
| O | O | O | Ph | OH | H | NH₂ | H | H | H | H |
| O | O | O | Me | OH | Me | NH₂ | H | H | H | H |
| O | O | O | i-Pr | OH | Me | NH₂ | H | H | H | H |
| O | O | O | t-Bu | OH | Me | NH₂ | H | H | H | H |
| O | O | O | Ph | OH | Me | NH₂ | H | H | H | H |
| O | O | O | Me | OH | H | NH₂ | Me | H | H | H |
| O | O | O | i-Pr | OH | H | NH₂ | Me | H | H | H |
| O | O | O | t-Bu | OH | H | NH₂ | Me | H | H | H |
| O | O | O | Ph | OH | H | NH₂ | Me | H | H | H |
| O | O | O | Me | OH | H | NH₂ | H | Me | H | H |
| O | O | O | i-Pr | OH | H | NH₂ | H | Me | H | H |
| O | O | O | t-Bu | OH | H | NH₂ | H | Me | H | H |
| O | O | O | Ph | OH | H | NH₂ | H | Me | H | H |
| O | O | O | Me | OH | H | NH₂ | CH₂Ph | H | H | H |
| O | O | O | i-Pr | OH | H | NH₂ | CH₂Ph | H | H | H |
| O | O | O | t-Bu | OH | H | NH₂ | CH₂Ph | H | H | H |
| O | O | O | Ph | OH | H | NH₂ | CH₂Ph | H | H | H |
| O | O | O | Me | NH₂ | H | H | H | H | H | H |
| O | O | O | i-Pr | NH₂ | H | H | H | H | H | H |
| O | O | O | Ph | NH₂ | H | H | H | H | H | H |
| O | O | O | Me | NH₂ | Me | H | H | H | H | H |
| O | O | O | i-Pr | NH₂ | Me | H | H | H | H | H |
| O | O | O | t-Bu | NH₂ | Me | H | H | H | H | H |
| O | O | O | Ph | NH₂ | Me | H | H | H | H | H |
| O | O | O | Me | NH₂ | H | H | Me | H | H | H |
| O | O | O | i-Pr | NH₂ | H | H | Me | H | H | H |
| O | O | O | t-Bu | NH₂ | H | H | Me | H | H | H |
| O | O | O | Ph | NH₂ | H | H | Me | H | H | H |
| O | O | O | Me | NH₂ | H | H | H | Me | H | H |
| O | O | O | i-Pr | NH₂ | H | H | H | Me | H | H |
| O | O | O | t-Bu | NH₂ | H | H | H | Me | H | H |
| O | O | O | Ph | NH₂ | H | H | H | Me | H | H |
| O | O | O | Me | NH₂ | H | H | CH₂Ph | H | H | H |
| O | O | O | i-Pr | NH₂ | H | H | CH₂Ph | H | H | H |
| O | O | O | t-Bu | NH₂ | H | H | CH₂Ph | H | H | H |
| O | O | O | Ph | NH₂ | H | H | CH₂Ph | H | H | H |
| O | O | O | Me | NH₂ | H | NH₂ | H | H | H | H |
| O | O | O | Me | H | Me | NH₂ | H | H | H | H |
| O | O | O | i-Pr | H | Me | NH₂ | H | H | H | H |
| O | O | O | t-Bu | H | Me | NH₂ | H | H | H | H |
| O | O | O | Ph | H | Me | NH₂ | H | H | H | H |

### Formula IV

In another embodiment of the present invention a structure of the formula (IV) is provided:

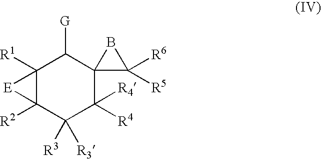

or its pharmaceutically acceptable salt, esters or prodrug, wherein:

B and E are independently O, S, NR⁷ or CR⁷R⁸;

G is OR¹¹, NR¹¹R¹² or SR¹¹;

R¹, R², R³, R⁴, R⁵ and R⁶ are independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, carbonyl, carboxylic acid, ester, carbamate, amide, amine, hydroxyl, alkoxy, nitro, cyano, azide, sulfonyl, sulfanyl, sulfinyl, sulfamoyl, phosphonyl, phosphinyl, phosphoryl, phosphine, a residue of a natural or synthetic amino acid, a residue of a natural or synthetic carbohydrate or XR⁹ (wherein X═O, S or NR¹⁰);

alternatively, one or more of R¹ and R², R² and R³ or R^{3′}, R³ or R^{3′} and R⁴ or R^{4′}, R⁴ or R^{4′} and R⁵, or R⁵ and R⁶, come together to form a bridged compound, preferably as a 3, 5, 6 or 7 membered ring, to form a cycloalkyl, cycloalkenyl, cycloalkynyl, aryl, heterocyclic, or heteroaryl;

R^{3′} and R^{4′} are independently hydrogen, halogen, azide, —OH, —OR⁹, —NH₂, —NHR⁹, —N(R⁹)₂, —SH, —SR⁹, —OC(O)R⁹, —OC(O)OR⁹, —OC(O)N(R⁹)₂, —NR⁹C(O)R⁹, —NR⁹C(O)OR⁹, —NR⁹C(O)N(R⁹)₂, —NR⁹SO₂R⁹, —SO₂N(R⁹)₂, —S(O)R⁹, —S(O)₂R⁹, —N—OR⁹, a residue of a natural or synthetic amino acid, or a residue of a natural or synthetic carbohydrate;

wherein two R⁹ groups on the same nitrogen may form an optionally substituted 3-8 membered heterocyclic or heteroaryl ring; and

each R⁷, R⁸, R⁹, R¹⁰, R¹¹ and R¹² is independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, a residue of a natural or synthetic amino acid or a residue of a natural or synthetic carbohydrate;

with the proviso that when R³ or R^{3′} are hydrogen, then R⁴ and R^{4′} are not hydrogen, and that when R⁴ or R^{4′} are hydrogen, then R³ and R^{3′} are not hydrogen.

In a first principle embodiment of formula (IV), G is OR¹¹. In one subembodiment, B is O and E is O. In another subembodiment, B is O and E is S. In still another subembodiment, B is O and E is NR⁷. In another subembodiment, B is O and E is CR⁷R⁸. In another subembodiment, B is S and E is O. In another subembodiment, B is S and E is S. In still another embodiment, B is S and E is NR⁷. In another subembodiment, B is S and E is CR⁷R⁸. In another subembodiment, B is NR⁷ and E is O. In another subembodiment, B is NR⁷ and E is S. In still another subembodiment, B is NR⁷ and E is NR⁷. In another subembodiment, B is NR⁷ and E is CR⁷R⁸. In yet another subembodiment, B is CR⁷R⁸ and E is O. In another subembodiment, B is CR⁷R⁸ and E is S. In another subembodiment, B is CR⁷R⁸ and E is NR⁷. In still another subembodiment, B is CR⁷R⁸ and E is CR⁷R⁸.

In another subembodiment, R^{3′} and R^{4′} are independently hydrogen, halogen, azide, —OH, —OR⁹, —NH₂, —NHR⁹, —N(R⁹)₂, —SH, —SR⁹, —OC(O)R⁹, —OC(O)OR⁹, —OC(O)N(R⁹)₂, a residue of a natural or synthetic amino acid, or a residue of a natural or synthetic carbohydrate; wherein two R⁹ groups on the same nitrogen may form an optionally substituted 3-8 membered heterocyclic or heteroaryl ring.

In another subembodiment, R^{3′} and R^{4′} are independently hydrogen, halogen, azide, —OH, —OR⁹, —NH₂, —NHR⁹, —N(R⁹)₂, —SH, —SR⁹, —OC(O)R⁹, —OC(O)OR⁹, —NR⁹C(O)R⁹, —NR⁹C(O)OR⁹, —NR⁹SO₂R⁹; wherein two R⁹ groups on the same nitrogen may form an optionally substituted 3-8 membered heterocyclic or heteroaryl ring.

In still another embodiment, R^{3′} and R^{4′} are independently hydrogen, —OH, —OR⁹, —NH₂, —NHR⁹, or —N(R⁹)₂; wherein two R⁹ groups on the same nitrogen may form an optionally substituted 3-8 membered heterocyclic or heteroaryl ring.

In still another embodiment, R¹, R², R³, R⁴, R⁵ and R⁶ are independently hydrogen, alkyl, alkenyl, alkynyl, cycloalkyl, cycloalkenyl, aryl, alkaryl, arylalkyl, heterocyclic, or heteroaryl.

In another subembodiment, R^{3′} and R^{4′} are independently hydrogen, —OH, —OR⁹, —NH₂, —NHR⁹, or —N(R⁹)₂; and R¹, R², R³ and R⁴ are independently hydrogen or alkyl.

In another subembodiment, R^{3′} and R^{4′} are independently —OH or —OR⁹; and R³ and R⁴ are independently hydrogen or alkyl.

In one embodiment, R^{3′} and R^{4′} are oriented in a cis-relationship to each other. In another embodiment, R^{3′} and R^{4′} are oriented in a trans-relationship to each other.
In still another subembodiment, R¹ and R⁴ are independently methyl, cyclohexyl, isopropyl or tert-butyl.
In a particular embodiment of the present invention, the compounds of the formula (IV) are the following species:

(IV)

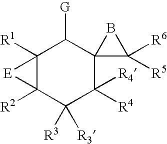

| A | B | D | R¹ | R^{3′} | R² | R^{4′} | R³ | R⁴ | R⁵ | R⁶ |
|---|---|---|---|---|---|---|---|---|---|---|
| O | O | O | Me | OH | H | OH | Me | H | H | H |
| O | O | O | i-Pr | OH | H | OH | Me | H | H | H |
| O | O | O | t-Bu | OH | H | OH | Me | H | H | H |
| O | O | O | Ph | OH | H | OH | Me | H | H | H |
| O | O | O | Me | OH | H | OH | H | Me | H | H |
| O | O | O | i-Pr | OH | H | OH | H | Me | H | H |
| O | O | O | t-Bu | OH | H | OH | H | Me | H | H |
| O | O | O | Ph | OH | H | OH | H | Me | H | H |
| O | O | O | Me | OH | H | OH | CH₂Ph | H | H | H |
| O | O | O | i-Pr | OH | H | OH | CH₂Ph | H | H | H |
| O | O | O | t-Bu | OH | H | OH | CH₂Ph | H | H | H |
| O | O | O | Ph | OH | H | OH | CH₂Ph | H | H | H |
| O | O | O | Me | OH | H | H | Me | H | H | H |
| O | O | O | i-Pr | OH | H | H | Me | H | H | H |
| O | O | O | t-Bu | OH | H | H | Me | H | H | H |
| O | O | O | Ph | OH | H | H | Me | H | H | H |
| O | O | O | Me | OH | H | H | CH₂Ph | H | H | H |
| O | O | O | i-Pr | OH | H | H | CH₂Ph | H | H | H |
| O | O | O | t-Bu | OH | H | H | CH₂Ph | H | H | H |
| O | O | O | Ph | OH | H | H | CH₂Ph | H | H | H |
| O | O | O | Me | H | H | OH | H | Me | H | H |
| O | O | O | i-Pr | H | H | OH | H | Me | H | H |
| O | O | O | t-Bu | H | H | OH | H | Me | H | H |
| O | O | O | Ph | H | H | OH | H | Me | H | H |
| O | O | O | Me | NH₂ | H | OH | Me | H | H | H |
| O | O | O | i-Pr | NH₂ | H | OH | Me | H | H | H |
| O | O | O | t-Bu | NH₂ | H | OH | Me | H | H | H |
| O | O | O | Ph | NH₂ | H | OH | Me | H | H | H |
| O | O | O | Me | NH₂ | H | OH | H | Me | H | H |
| O | O | O | i-Pr | NH₂ | H | OH | H | Me | H | H |
| O | O | O | t-Bu | NH₂ | H | OH | H | Me | H | H |
| O | O | O | Ph | NH₂ | H | OH | H | Me | H | H |
| O | O | O | Me | NH₂ | H | OH | CH₂Ph | H | H | H |
| O | O | O | i-Pr | NH₂ | H | OH | CH₂Ph | H | H | H |
| O | O | O | t-Bu | NH₂ | H | OH | CH₂Ph | H | H | H |
| O | O | O | Ph | NH₂ | H | OH | CH₂Ph | H | H | H |
| O | O | O | Me | OH | H | NH₂ | Me | H | H | H |
| O | O | O | i-Pr | OH | H | NH₂ | Me | H | H | H |
| O | O | O | t-Bu | OH | H | NH₂ | Me | H | H | H |
| O | O | O | Ph | OH | H | NH₂ | Me | H | H | H |
| O | O | O | Me | OH | H | NH₂ | H | Me | H | H |
| O | O | O | i-Pr | OH | H | NH₂ | H | Me | H | H |
| O | O | O | t-Bu | OH | H | NH₂ | H | Me | H | H |
| O | O | O | Ph | OH | H | NH₂ | H | Me | H | H |
| O | O | O | Me | OH | H | NH₂ | CH₂Ph | H | H | H |
| O | O | O | i-Pr | OH | H | NH₂ | CH₂Ph | H | H | H |
| O | O | O | t-Bu | OH | H | NH₂ | CH₂Ph | H | H | H |
| O | O | O | Ph | OH | H | NH₂ | CH₂Ph | H | H | H |
| O | O | O | Me | NH₂ | H | H | Me | H | H | H |
| O | O | O | i-Pr | NH₂ | H | H | Me | H | H | H |
| O | O | O | t-Bu | NH₂ | H | H | Me | H | H | H |
| O | O | O | Ph | NH₂ | H | H | Me | H | H | H |
| O | O | O | Me | NH₂ | H | H | CH₂Ph | H | H | H |
| O | O | O | i-Pr | NH₂ | H | H | CH₂Ph | H | H | H |
| O | O | O | t-Bu | NH₂ | H | H | CH₂Ph | H | H | H |
| O | O | O | Ph | NH₂ | H | H | CH₂Ph | H | H | H |
| O | O | O | Me | H | H | NH₂ | H | Me | H | H |
| O | O | O | i-Pr | H | H | NH₂ | H | Me | H | H |
| O | O | O | t-Bu | H | H | NH₂ | H | Me | H | H |
| O | O | O | Ph | H | H | NH₂ | H | Me | H | H |

### Formula V

In another embodiment of the present invention a structure of the formula (V) is provided:

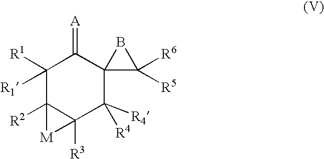

or its pharmaceutically acceptable salt, esters or prodrug, wherein:

A, B and M are independently O, S, NR⁷ or CR⁷R⁸;

R¹, R^{1′} R², R³, R⁴, R^{4′} R⁵ and R⁶ are independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, carbonyl, carboxylic acid, ester, carbamate, amide, amine, hydroxyl, alkoxy, nitro, cyano, azide, sulfonyl, sulfanyl, sulfinyl, sulfamoyl, phosphonyl, phosphinyl, phosphoryl, phosphine, a residue of a natural or synthetic amino acid, a residue of a natural or synthetic carbohydrate or XR⁹ (wherein X═O, S or NR¹⁰);

alternatively, one or more of R¹ or R^{1′} and R², R² and R³, R³ and R⁴ or R^{4′}, R⁴ or R^{4′} and R⁵, or R⁵ and R⁶, come together to form a bridged compound, preferably as a 3, 5, 6 or 7 membered ring, to form a cycloalkyl, cycloalkenyl, cycloalkynyl, aryl, heterocyclic, or heteroaryl; and

each R⁷, R⁸, R⁹ and R¹⁰ is independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, a residue of a natural or synthetic amino acid or a residue of a natural or synthetic carbohydrate;

with the proviso that when R¹ or R^{1′} are hydrogen, then R⁴ and R^{4′} are not hydrogen, and that when R⁴ or R^{4′} are hydrogen, then R¹ and R^{1′} are not hydrogen.

In a first principle embodiment of formula (V), A is O. In one subembodiment, B is O and M is O. In another subembodiment, B is O and M is S. In still another subembodiment, B is O and M is NR⁷. In another subembodiment, B is O and M is CR⁷R⁸. In another subembodiment, B is S and M is O. In another subembodiment, B is S and M is S. In still another embodiment, B is S and M is NR⁷. In another subembodiment, B is S and M is CR⁷R⁸. In another subembodiment, B is NR⁷ and M is O. In another subembodiment, B is NR⁷ and M is S. In still another subembodiment, B is NR⁷ and M is NR⁷. In another subembodiment, B is NR⁷ and M is CR⁷R⁸. In yet another subembodiment, B is CR⁷R⁸ and M is O. In another subembodiment, B is CR⁷R⁸ and M is S. In another subembodiment, B is CR⁷R⁸ and M is NR⁷. In still another subembodiment, B is CR⁷R⁸ and M is CR⁷R⁸.

In still another subembodiment, R¹, R^{1′} R², R³, R⁴, R^{4′} R⁵ and R⁶ are independently hydrogen, alkyl, alkenyl, alkynyl, cycloalkyl, cycloalkenyl, aryl, alkaryl, arylalkyl, heterocyclic, or heteroaryl.

In another subembodiment, R⁵ and R⁶ are hydrogen.

In still another subembodiment, R¹ is methyl, cyclohexyl, isopropyl or tert-butyl and R² is hydrogen.

### Formula VI

In another embodiment of the present invention a structure of the formula (VI) is provided:

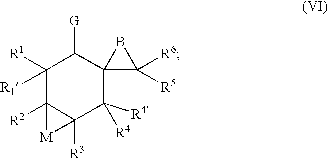

or its pharmaceutically acceptable salt, esters or prodrug, wherein:

B and M are independently O, S, NR⁷ or CR⁷R⁸;

G is OR¹¹, NR¹¹R¹² or SR¹¹;

R¹, R^{1′}, R², R³, R⁴, R^{4′} R⁵ and R⁶ are independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, carbonyl, carboxylic acid, ester, carbamate, amide, amine, hydroxyl, alkoxy, nitro, cyano, azide, sulfonyl, sulfanyl, sulfinyl, sulfamoyl, phosphonyl, phosphinyl, phosphoryl, phosphine, a residue of a natural or synthetic amino acid, a residue of a natural or synthetic carbohydrate or XR⁹ (wherein X═O, S or NR¹⁰);

alternatively, one or more of R¹ or R^{1′} and R², R² and R³, R³ and R⁴ or R^{4′}, R⁴ or R^{4′} and R⁵, or R⁵ and R⁶, come together to form a bridged compound, preferably as a 3, 5, 6 or 7 membered ring, to form a cycloalkyl, cycloalkenyl, cycloalkynyl, aryl, heterocyclic, or heteroaryl; and

each R⁷, R⁸, R⁹, R¹⁰, R¹¹ and R¹² is independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, a residue of a natural or synthetic amino acid or a residue of a natural or synthetic carbohydrate;

with the proviso that when R¹ or R^{1′} are hydrogen, then R⁴ and R^{4′} are not hydrogen, and that when R⁴ or R^{4′} are hydrogen, then R¹ and R^{1′} are not hydrogen.

In a first principle embodiment of formula (VI), G is OR¹¹. In one subembodiment, B is O and M is O. In another subembodiment, B is O and M is S. In still another subembodiment, B is O and M is NR⁷. In another subembodiment, B is O and M is CR⁷R⁸. In another subembodiment, B is S and M is O. In another subembodiment, B is S and M is S. In still another embodiment, B is S and M is NR⁷. In another subembodiment, B is S and M is CR⁷R⁸. In another subembodiment, B is NR⁷ and M is O. In another subembodiment, B is NR⁷ and M is S. In still another subembodiment, B is NR⁷ and M is NR⁷. In another subembodiment, B is NR⁷ and M is CR⁷R⁸. In yet another subembodiment, B is CR⁷R⁸ and M is O. In another subembodiment, B is CR⁷R⁸ and M is S. In another subembodiment, B is CR⁷R⁸ and M is NR⁷. In still another subembodiment, B is CR⁷R⁸ and M is CR⁷R⁸.

In still another subembodiment, R¹, R^{1′} R², R³, R⁴, R^{4′} R⁵ and R⁶ are independently hydrogen, alkyl, alkenyl, alkynyl, cycloalkyl, cycloalkenyl, aryl, alkaryl, arylalkyl, heterocyclic, or heteroaryl.

In another subembodiment, R⁵ and R⁶ are hydrogen.
In still another subembodiment, R¹ is methyl, cyclohexyl, isopropyl or tert-butyl and R² is hydrogen.

### II. STEREOISOMERS

The compounds of the invention can be provided in any stereo or regioisomeric configuration. Furthermore, each of the compounds which have asymmetric centers can exist as a pair of enantiomers.

In one embodiment, R^{1′} and R^{2′} are in a cis-configuration relative to each other. In another embodiment, R^{1′} and R^{2′} are in a trans-configuration relative to each other. In another embodiment, R^{3′} and R^{4′} are in a cis-configuration relative to each other. In still another embodiment, R^{3′} and R⁴ are in a trans-configuration relative to each other.

In general, if there are n asymmetric centers, a total of 2ⁿ possible stereochemical isomers will exist. In addition each three membered ring may be oriented either out of the plane or into the plane of the page as drawn, resulting in different stereochemical configurations. All stereoisomeric forms of the compounds of formula I-VI including specific diastereomers and enantiomers are embraced by the invention. The example below illustrates the possible stereochemical isomers for the compound of formula I based on the configuration of the groups R¹, R^{1′}, R², R^{2′}, R³, R^{3′}, R⁴ and R^{4′}.

For example, with respect only to the substituents R¹, R^{1′}, R² and R^{2′} for the compounds of formula I, if substituent R¹ is different than R^{1′} and substituent R² is different than R^{2′}, the four optical isomers shown below are possible (two sets of enantiomers and diastereomers). Of course, additional stereoisomers are possible when taking into account the stereochemical configuration of ring B and ring D, as described above. All of these stereoisomeric forms are encompassed by the invention.

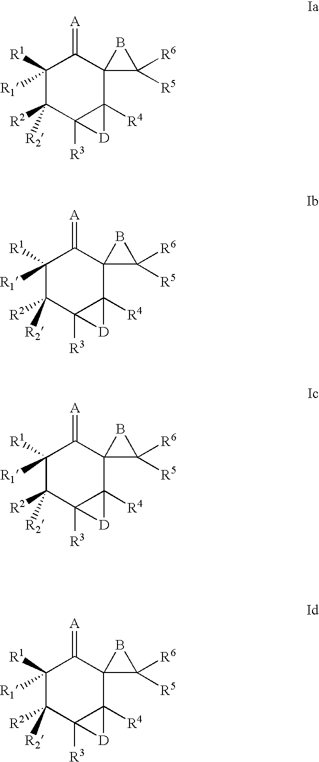

### III. DEFINITIONS

The terms used herein are intended to have their ordinary meaning in the art, unless indicated otherwise.

It should be understood that the various possible stereoisomers of the groups mentioned above and herein are within the meaning of the individual terms and examples, unless otherwise specified. As an illustrative example, “1-methyl-butyl” exists in both the (R) and the (S) form, thus, both (R)-1-methyl-butyl and (S)-1-methyl-butyl is covered by the term “1-methyl-butyl,” unless otherwise specified. Several biological compounds are designed by the (D) and the (L) form, rather than the (R) and the (S) form, respectively, based on the stereochemistry around the 4′ carbon. As an another illustrative example, “glycine” exists in both the (D) and the (L) form; therefore, both (D)-glycine and (L)-glycine are covered by the term “glycine” unless otherwise specified.

As used herein, the term “isolated enantiomer” refers to a composition that includes at least approximately 95% to 100%, or more preferably, over 97% of a single enantiomer of that compound.

As used herein, the term “substantially free of” or “substantially in the absence of” refers to a composition that includes at least 85 or 90% by weight, preferably 95% to 98% by weight, and even more preferably 99% to 100% by weight, of the designated enantiomer of that compound.

The term “independently” is used herein to indicate that the variable that is independently applied varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

The term “alkyl,” as used herein, unless otherwise specified, refers to optionally substituted saturated straight, branched, or cyclic, primary, secondary, or tertiary hydrocarbon, typically of C₁ to C₁₈ and in certain embodiment of C₁ to C₁₀ or of C₁ to C₆, and specifically includes methyl, ethyl, propyl, isopropyl, butyl, isobutyl, t-butyl, pentyl, cyclopentyl, isopentyl, neopentyl, hexylisohexyl, cyclohexyl, cyclohexylmethyl, 3-methylpentyl, 2,2-dimethylbutyl and 2,3-dimethylbutyl. The alkyl group can be optionally substituted with one or more moieties, including alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thiol, imine, sulfonic acid, sulfate, sulfonyl, sulfanyl, sulfinyl, sulfamoyl, ester, carboxylic acid, amide, phosphonyl, phosphinyl, phosphoryl, phosphine, thioester, thioether, acid halide, anhydride, oxime, hydrozine, carbamate, phosphonic acid, phosphate, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “lower alkyl,” as used herein, and unless otherwise specified, refers to optionally substituted C₁ to C₄ saturated straight, branched, or if appropriate, a cyclic (for example, cyclopropyl) alkyl group, including both substituted and unsubstituted forms.

The term “alkylene” refers to optionally substituted saturated hydrocarbyldiyl radical of straight or branched configuration, including but not limited to those that have from one to ten carbon atoms. Included within the scope of this term are methylene, 1,2-ethane-diyl, 1,1-ethane-diyl, 1,3-propane-diyl, 1,2-propane-diyl, 1,3-butane-diyl, 1,4-butane-diyl and the like. The alkylene group or other divalent moiety disclosed herein can be optionally substituted with one or more moieties, including alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamoyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art or organic synthesis. Suitable protecting groups are described, for example, in Greene, et al., “Protective Groups in Organic Synthesis,” John Wiley and Sons, Second Edition, 1991, hereby incorporated by reference.

The term “aryl,” as used herein, and unless otherwise specified, refers to optionally substituted C₆ to C₁₄ aromatic ring. Aryl groups include, but are not limited to, phenyl, biphenyl, or naphthyl. The aryl group can be optionally substituted with one or more moieties. Examples of substituents include alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfate, sulfonyl, sulfanyl, sulfinyl, sulfamoyl, ester, carboxylic acid, amide, phosphate, phosphonyl, phosphinyl, phosphoryl, phosphine, thioester, thioether, acid halide, anhydride, oxime, hydrozine, carbamate, phosphonic acid, phosphonateeither unprotected, or protected as necessary, as known to those skilled in the art, for example, as taught in Greene, et al., “Protective Groups in Organic Synthesis,” John Wiley and Sons, Second Edition, 1991.

The term “aralkyl,” as used herein, and unless otherwise specified, refers to an optionally substituted aryl group as defined above linked to the molecule through an alkyl group as defined above. The term alkaryl or alkylaryl as used herein, and unless otherwise specified, refers to an alkyl group as defined above linked to the molecule through an aryl group as defined above. In each of these groups, the alkyl and aryl groups can be optionally substituted as describe above sulfamoyl Specifically included within the scope of the term aryl are phenyl; naphthyl; phenylmethyl; phenylethyl; 3,4,5-trihydroxyphenyl; 3,4,5-trimethoxyphenyl; 3,4,5-triethoxyphenyl; 4-chlorophenyl; 4-methylphenyl; 3,5-di-tertiarybutyl-4-hydroxyphenyl; 4-fluorophenyl; 4-chloro-1-naphthyl; 2-methyl-1-naphthylmethyl; 2-naphthylmethyl; 4-chlorophenylmethyl; 4-tertiarybutylphenyl; 4-tertiarybutylphenylmethyl and the like.

The term “halo” or “halogen,” as used herein includes chloro, bromo, iodo and fluoro.

The term heteroatom, as used herein, refers to oxygen, sulfur, nitrogen or phosphorus.

The term “alkylamino” or “arylamino” refers to an amino group that has one or two alkyl or aryl substituents, respectively.

The term “alkoxy,” as used herein, and unless otherwise specified, refers to a moiety of the structure —O-alkyl, wherein alkyl is as defined above.

The term “acyl” refers to moiety of the formula —C(O)R′, wherein R′ is alkyl; aryl, alkaryl, aralkyl, heterocyclic, alkoxyalkyl including methoxymethyl; arylalkyl including benzyl; aryloxyalkyl, such as phenoxymethyl; aryl including phenyl optionally substituted with halo groups C₁ to C₄ alkyl or C₁ to C₄ alkoxy or the residue of an amino acid.

As used herein, a “leaving group” means a functional group that is cleaved from the molecule to which it is attached under appropriate conditions.

The term “heteroaryl”, as used herein, refers to an aromatic that includes at least one sulfur, oxygen, nitrogen or phosphorus in the aromatic ring. The term heterocyclic refers to a nonaromatic cyclic group wherein there is at least one heteroatom, such as oxygen, sulfur, nitrogen or phosphorus in the ring. Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, or imidazole. The heteroaryl group can be optionally substituted as described above for aryl. The heterocyclic group can be optionally substituted with one or more moieties, including but not limited to, alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamoyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference. The heteroaryl can be partially or totally hydrogenated as desired. As a nonlimiting example, dihydropyridine can be used in place of pyridine. Functional oxygen and nitrogen groups on the heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenesulfonyl.

The term “amino acid” includes naturally occurring and synthetic amino acids, and includes but is not limited to, alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl and histidinyl.

The term “ether,” as used herein, refers to oxygen that is disubstituted with independent alkyl groups or two alkyl groups that together formed a ring or a bridge. Some non-limiting examples include 4-(tetrahydrobenzimidazol-1-yl)butoxy, 5-(tetrahydrobenzimidazol-1-yl)pentoxy, ethoxy, n-propoxy or isoproproxy. The ethers also can be optionally substituted with one or more moieties, including alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamoyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “amide,” as used herein, refers to a carbonyl moiety wherein the non-alkyl moiety is formed from an amine Some non-limiting examples are formylamino, acetylamino, propionylamino, butanoylamino, isobutanoylamino, pentanoylamino, 3-methyl-butanoylamino, hexanoylamino, methoxycarbonylamino, ethoxycarbonylamino, n-propoxycarbonylamino, isopropoxycarbonylamino, benzamido, cyclopentylcarbonyl-amido, cyclohexylcarbonylamido, cycloheptylcarbonyl-amido, phenylacetylamido, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “sulfamoyl” is a hexavalent sulfur covalently bound to at least two oxygens and a nitrogen. Some non-limiting examples include methanesulphonylamino, ethanesulphonylamino, n-propanesulphonylamino, isopropanesulphonylamino, n-butane-sulphonylamino, N-ethyl-phenylmethanesulphonylamido, N-ethyl-2-phenylethane-sulphonylamido, N-ethyl-3-phenylpropanesulphonylamido, N-ethyl-naphthalen-1-yl-sulphonamido or N-ethyl-naphthalen-2-yl-sulphonylamido. The sulfamoyl group also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamoyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “thio” or “thiol” refers to a sulfur covalently bound to a hydrogen or a carbon based group. Some non-limiting examples include methylmercapto, ethylmercapto, n-propylmercapto, isopropylmercapto or n-butylmercapto, ethylthio, n-propylthio or isopropylthio group. The thio group also can be optionally substituted with one or more moieties, including but not limited to, alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamoyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “ester” refers to a carbonyl flanked by an alkoxy group and a carbon based group. Some non-limiting examples include hydroxycarbonyl, methoxycarbonyl, ethoxycarbonyl, n-propyloxycarbonyl, isopropyloxycarbonyl, n-butyloxycarbonyl, isobutyloxycarbonyl, tert-butyloxycarbonyl or 1-(cinnamyloxycarbonyloxy)-ethoxy-carbonyl. The ester group also can be optionally substituted with one or more moieties, including but not limited to, alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamoyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “urethane” or “carbamate” refers to —OC(O)NR⁴R⁵ in which R⁴ and R⁵ are independently selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. Aryl groups in the carbamide optimally comprise a phenyl group. The term “lower carbamide” refers to a carbamide group in which the non-carbonyl moiety is a lower alkyl. The carbamide group also can be optionally substituted with one or more moieties, including but not limited to, alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamoyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “carbohydrate,” used herein refers to mono, di, tri, oligo, and poly saccharides consisting of furanose and pyranose sugars such as threose, ribulose, ketose, gentiobiose, aldose, aldotetrose, aldopentose, aldohexose, ketohexose, ketotetrose, ketopentose, erythrose, threose, ribose, deoxyribose, arabinose, xylose, lyxose, allose, altrose, glucose, mannose, gulose, idose, glactose, talose, erythrulose, ribulose, xylulose, psicose, fructose, sorbose, tagatose, dextrose, maltose, lactose, sucrose, or cellulose. The carbohydrate moiety as disclosed herein can be optionally substituted with one or more moieties, including but not limited to, alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamoyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “alkylheteroaryl” refers to an alkyl group substituted by a heteroaryl substituent.

The term “host,” as used herein, refers to a multicellular organism which is at risk of or exhibiting symptoms of an autoimmune, inflammatory or hyper- or abnormally proliferative disorder. Typically the host is a human. The term host specifically refers to animals, in particular, primates (including chimpanzees) and humans, in which autoimmune and inflammatory disorders or disorders of abnormal cell proliferation occur. In most animal applications of the present invention, the host is a human patient in need of treatment or prevention of symptoms of a disorder as described herein. Veterinary applications, in certain indications, however, are clearly anticipated by the present invention (such as chimpanzees).

### IV. PHARMACEUTICALLY ACCEPTABLE SALT FORMULATIONS

Modifications of the active compound can affect the bioavailability and rate of metabolism of the active species, thus providing control over the delivery of the active species. Further, the modifications can affect the activity of the compound, in some cases increasing the activity over the parent compound. This can easily be assessed by preparing the derivative and testing its activity according to the methods described herein, or other method known to those skilled in the art.

In cases where compounds are sufficiently basic or acidic to form stable nontoxic acid or base salts, administration of the compound as a pharmaceutically acceptable salt may be appropriate. The term “pharmaceutically acceptable salts” or “complexes” refers to salts or complexes that retain the desired biological activity of the compounds of the present invention and exhibit minimal undesired toxicological effects.

Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, a-ketoglutarate and a-glycerophosphate. Suitable inorganic salts may also be formed, including, sulfate, nitrate, bicarbonate and carbonate salts. Alternatively, the pharmaceutically acceptable salts may be made with sufficiently basic compounds such as an amine with a suitable acid affording a physiologically acceptable anion. Alkali metal (for example, sodium, potassium or lithium) or alkaline earth metal (for example calcium) salts of carboxylic acids can also be made.

Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalcturonic acid; (b) base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like. Also included in this definition are pharmaceutically acceptable quaternary salts known by those skilled in the art, which specifically include the quaternary ammonium salt of the formula —NR⁺A⁻, wherein R is as defined above and A is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of this invention possess anti-inflammatory activity, or are metabolized to a compound that exhibits such activity.

Any of the compounds described herein can be administered as a prodrug to increase the activity, bioavailability, stability or otherwise alter the properties of the compound. A number of prodrug ligands are known. In general, alkylation, acylation or other lipophilic modification of the compound will increase the stability of the compound. Examples of substituent groups that can replace one or more hydrogens on the compound are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed compounds to achieve a desired effect.

### V. TREATMENT OF MEDICAL DISORDERS

The immune system functions as the body's major defense against diseases caused by invading organisms. The immune system's ability to distinguish the body's normal tissues, or self, from foreign or cancerous tissue, or non-self, is an essential feature of normal immune system function. The loss of recognition of a particular tissue as self and the subsequent immune response directed against that tissue produce serious illness.

The immune response is initiated by the interaction of an antigen with macrophages and surface antibodies on B cells. Activated macrophages secrete interleukin-1 (IL-1) and tumor necrosis factor (TNF), and display the processed antigen on the cell surface together with a major antihistocompatibility antigen. Importantly, IL-1 activates T cells that release a series of lymphokines including interleukin-2 (IL-2) that activate the proliferation of T cells and cytotoxic lymphocytes. Inflammatory reactions differ not only as to the nature of the triggering event, but also in the types of cells mediating the response and in the biochemical nature of the end effectors. In particular, inflammation mediated by monocyte/macrophage activity can result in severe chronic or fatal conditions.

Leukocyte activation leads to the release of degradative enzymes, the generation of reactive oxygen species and the biosynthesis of locally acting pro-inflammatory autacoids. Among the latter, oxygenated metabolites of arachidonic acid are recognized major products of leukocyte activation. The arachidonate lipoxygenase (LO) family of enzymes catalyze the formation of highly potent biologic mediators in leukocytes and platelets.

Each element in the cascade of the immune response may be considered as a potential site for pharmacological intervention. Numerous immunosuppressive agents have been identified that, for example, inhibit synthesis and release of IL-1.

### Autoimmune and Inflammatory Diseases

The compounds of the present invention can be used to treat disorders mediated by a member of the lipoxygenase family (LO). Dysfunction in LO production is implicated in a wide variety of disease states that can be treated, prevented or ameliorated by administration of a compound as described herein to a host suffering from, or at risk of suffering from, the disease, include but are not limited to arthritis, asthma, dermatitis, psoriasis, cystic fibrosis, post transplantation late and chronic solid organ rejection, multiple sclerosis, systemic lupus erythematosis, inflammatory bowel diseases, autoimmune diabetes, diabetic retinopathy, rhinitis, ischemia-reperfusion injury, post-angioplasty restenosis, chronic obstructive pulmonary disease (COPD), glomerulonephritis, Graves disease, gastrointestinal allergies, and conjunctivitis.

Nonlimiting examples of arthritis include rheumatoid (such as soft-tissue rheumatism and non-articular rheumatism, fibromyalgia, fibrositis, muscular rheumatism, myofascil pain, humeral epicondylitis, frozen shoulder, Tietze's syndrome, fascitis, tendinitis, tenosynovitis, bursitis), juvenile chronic, spondyloarthropaties (ankylosing spondylitis), osteoarthritis, hyperuricemia and arthritis associated with acute gout, chronic gout and systemic lupus erythematosus.

Certain endothelial disorders mediated by LO that can be treated, prevented or ameliorated by administration of a compound as described herein to a host suffering from, or at risk of suffering from, the disease include psoriasis, eczematous dermatitis, Kaposi's sarcoma as well as proliferative disorders of smooth muscle cells.

In yet another embodiment, the compounds disclosed herein can be administered to treat inflammatory conditions that are mediated by mononuclear leukocytes.

In another embodiment, a method of treatment or prophylaxis of Sjögren's syndrome is provided comprising administering a compound of the invention to a host in need thereof. Sjögren's syndrome is an autoimmune disorder in which immune cells attack and destroy the exocrine glands that produce tears and saliva.

In another embodiment, a method of treatment of polymyositis or dermatomyositis is provided comprising administering a compound of the invention to a host in need thereof. Polymyositis is a disease of muscle featuring inflammation of the muscle fibers. The cause of the disease is not known. It begins when white blood cells, the immune cells of inflammation, spontaneously invade muscles. The muscles affected are typically those closest to the trunk or torso. This results in weakness that can be severe. Polymyositis is a chronic illness with periods of increased symptoms, called flares or relapses, and minimal or no symptoms, known as remissions. Polymyositis can be associated with skin rash and is then referred to as “dermatomyositis.” It also can affect other areas of the body and is, therefore, referred to as a systemic illness. Occasionally, it is associated with cancer or with other diseases of connective tissue (see systemic lupus erythematosus, scleroderma and rheumatoid arthritis).

In another embodiment, a method of treatment or prophylaxis of autoimmune hepatitis is provided comprising administering a compound of the invention to a host in need thereof.

In another embodiment, a method of treatment or prophylaxis of ulcerative colitis, Crohn's disease is provided comprising administering a compound of the invention to a host in need thereof.

In another embodiment, a method of treatment or prophylaxis of psoriatic arthritis is provided comprising administering a compound of the invention to a host in need thereof.

In another embodiment, a method of treatment or prophylaxis of vasculitis (temporal arteritis, poly arteritis nodosa, Wegener's granulomatosis, Takayasu's arteritis) is provided comprising administering a compound of the invention to a host in need thereof.

In another embodiment, a method of treatment or prophylaxis of Sarcoidosis is provided comprising administering a compound of the invention to a host in need thereof. Sarcoidosis, also called sarcoid (from the Greek sarx, meaning “flesh”) or Besnier-Boeck disease, is a multisystem disorder characterized by non-caseating granulomas (small inflammatory nodules). It most commonly arises in young adults. The cause of the disease is still unknown. Virtually any organ can be affected; however, granulomas most often appear in the lungs or the lymph nodes. Symptoms usually appear gradually but can occasionally appear suddenly. The clinical course generally varies and ranges from asymptomatic disease to a debilitating chronic condition that may lead to death.

In another embodiment, a method of treatment or prophylaxis of scleroderma is provided comprising administering a compound of the invention to a host in need thereof. Scleroderma is a chronic autoimmune disease characterized by a hardening or sclerosis in the skin or other organs. The localized type of the disease, known as “morphea”, while disabling, tends not to be fatal. The systemic type or systemic sclerosis, the generalized type of the disease, can be fatal, as a result of heart, kidney, lung or intestinal damage.

In one embodiment, the compounds of the present invention are administered for the treatment or prophylaxis of tissue or organ transplant rejection in a host at risk of or suffering from the rejection. Treatment and prophylaxis of organ or tissue transplant rejection includes, but are not limited to treatment of recipients of heart, lung, combined heart-lung, liver, kidney, pancreatic, skin, spleen, small bowel, or corneal transplants. The compounds can also be used in the prevention or treatment of graft-versus-host disease, such as sometimes occurs following bone marrow transplantation.

Rheumatoid arthritis (RA) is a chronic inflammatory disease characterized by hyperplasia of the synovial lining cells which cartilage destruction in the RA joint (Harris E D J. “Rheumatoid arthritis. Pathophysiology and implications for therapy” N Engl J Med 1990 322:1277-89; Volin M V and Koch A E. “Cell cycle implications in the pathogenesis of rheumatoid arthritis” Frontiers in Bioscience 2000 5:D594-601;). The altered rates of proliferation and apoptosis of RA synovial cells result in the hyperplasia of synovial tissue and in concert with the chronic inflammatory environment ultimately lead to the destruction of the RA joint. In certain embodiments, the compounds of the invention are used in the treatment or prophylaxis of rheumatoid arthritis alone or in combination with another active agent in a host suffering from, or at risk of suffering from, the condition.

In certain other embodiments, a method is provided to treat, ameliorate or prevent a form of psoriasis by administering a compound of the invention alone or in combination with another active agent to a host suffering from or at risk of suffering from the condition. In some embodiments, the psoriasis is selected from, plaque psoriasis, guttate psoriasis, inverse psoriasis, seborrheic psoriasis, nail psoriasis, generalized erythrodermic psoriasis (also called psoriatic exfoliative erythroderm), pustular psoriasis, and Von Zumbusch psoriasis.

### Treatment of Proliferative Disorders and Cancer

The compounds of the present invention can be used in the treatment or prophylaxis of cancer as well as other abnormal cell proliferation-associated diseases by administration to a host suffering from or at risk of suffering from the diseases.

Abnormal proliferation of vascular smooth muscle cells has been implicated in forms of human pathogenesis other than cancer. Benign tumors are characterized by abnormal cell proliferation but are not malignant, recurrent, invasive or progressive. Yet, due to metabolic effects or critical location (e.g., the brain), certain benign tumors can have devastating consequences.

Abnormal proliferation of smooth muscle cells is involved in atherosclerosis and restenosis. (Sanz-Gonzalez S M, Poch E, Perez-Roger I, Diez-Juan A, Ivorra C, Andres V. “Control of vascular smooth muscle cell growth by cyclin-dependent kinase inhibitory proteins and its implication in cardiovascular disease” Front Biosci. 2000 1:5:D619-28; Andres V. “Control of vascular smooth muscle cell growth and its implication in atherosclerosis and restenosis (review)” Int J Mol. Med. 1998 2(1):81-9;). Vascular smooth muscle cells within the medial layer of elastic arteries proliferate in response to physiological and pathological stimuli causing neointimal thickening during spontaneous atherosclerosis and vessel renarrowing (restenosis) after angioplasty. Abnormal cell proliferation is also involved in lymphangiomyomatosis (LAM); a progressive lung disease characterized by overgrowth of smooth muscle inside the lung. Most patients die within 15 years of symptom onset (Reid J K, Rees H, Cockcroft D. “Long term survival in a patient with pulmonary lymphangioleiomyomatosis” Can Respir J. 2002 9(5):342-6).

Mastocytosis encompasses a range of disorders characterized by over-proliferation and accumulation of tissue mast cells. It is a disease of complex etiology. The skin is frequently directly involved in mastocytosis (cutaneous mastocytosis or CM) but the skeleton, gastrointestinal tract, bone marrow, and central nervous system may also be involved. Aggressive mastocytosis is a form of systemic mast cell disease characterized by organ infiltration, bone lesions, eosinophilia and lymphadenopathies.

Mesangial cell proliferation is a prominent feature of many human glomerular diseases including IgA nephropathy, membranoproliferative glomerulonephritis (GN), lupus nephritis and diabetic nephropathy (Jefferson J, Johnson R. “Experimental mesangial proliferative glomerulonephritis (the anti-Thy-1.1 model)” J Nephrol 1999 12: 297-307).

Psoriasis is a relatively common chronic (and non-infectious) skin disease in which epidermal regeneration has become unregulated. It is generally believed that psoriasis is caused by impairment in the immune system, enzymes, and other biochemical substances that regulate skin cell division. One or more genetic abnormalities maybe involved. There are a variety of types of psoriasis, with the most common known as plaque psoriasis.

### Cancer

Cancer represents a diverse class of diseases that occur occurs when a cell escapes from regulated growth control. In general, cancers are divided into four types based on their cell of origin: carcinomas, lymphomas, leukemias and sarcomas. Cancers may be metastatic, meaning cancer cells spread from where they arose (the primary site) to another part (the metastatic site). The most common causes of cancer death are malignancies of the lung, colorectum, breast, prostate, hematolymphoid system, urinary tract, and pancreas.

Cancer is a diverse group of diseases, affecting various tissues and organs of the body. Cancer cells are malignant, fast-growing cells that can metastasize. Different types of carcinomas, sarcomas, lymphomas, and leukemias are typically named using different prefixes represents the cell type including adeno- (gland), chondro- (cartililege), erythro-(red blood cell); hemangio- (blood vessel), hepato- (liver), lipo- (fat), lympho-(lymphocyte), melano- (pigment cell), myelo- (bone marrow), myo- (muscle) and osteo-(bone), as mentioned above.

Carcinomas are tumors (i.e., neoplasms) arising from epithelial tissue, such as glands, breast, skin, and linings of the urogenital, digestive, and respiratory systems. Breast cancer is a particularly most common form of carcinoma that originates in the ducts or lobules of the breast. Invasive cancer cells that spread (i.e., by moving through blood or lymphatic vessels) beyond the breast area to other parts of the body (i.e., bone or lung), are known as metastatic breast cancer. Prostate cancer is the most common male malignancy in developed countries and the second leading cause of cancer mortality (Rebillard X, Tretarre B, Villers A. “The epidemiology of prostate cancer” Rev Prat. 2003 53(20):2224-8; Greenlee R T et al. “Cancer Statistics, 2001” Cancer J. Clin. 2001 51:15-38). While prostate cancer is typically not a fatal disease, it can cause a variety of uncomfortable symptoms that severely impact quality of life.

Lymphomas are cancers that originate in the lymph nodes and spleen that are characterized by the excessive production of lymphocytes. Lymphomas are generally grouped to include Non-Hodgkin's lymphoma and Hodgkin's disease, with the former much more prevalent than the latter. Hodgkin's disease is defined histopathologically by the presence of the malignant Reed Sternberg cells in the cancerous area. Non-Hodgkin's lymphoma refers to a group of nearly thirty lymphomas classified by lymphatic cell type and growth rate (Harris N L, Jaffe E S, Kiebold J, Flandrin G, Muller-Hermelink H K, Vardiman J. “Lymphoma classification—from controversy to consensus: the REAL and WHO Classification of lymphoid neoplasms” Ann Oncol. 2000; 11(suppl 1):S3-S10).

Leukemias originate in the bone marrow and are generally classified as lymphocytic or myeloid, depending on the type of leukocyte involved. Leukemias are further classified as acute (i.e., a rapidly progressing disease that involves immature leukocytes) or chronic (i.e, a slower proliferation involving mature white cells). Myeloma is highly related to leukemia as a cancer of plasma cells; a type of white blood cell found throughout the body but primarily in the bone marrow.

Sarcomas are the least common form of cancer (2%). Some originate in bone while others originate in the soft tissues including muscles, tendons, fibrous tissues, fat, blood vessels nerves, and synovial tissues (tissues around joints). Clinically relevant malignant sarcomas include osteosarcoma (cancerous tumor of the bone), neurofibrosarcomas (malignant tumors of nerve sheath origin), liposarcomas (malignant lesions of adipose tissue), rhabdomyosarcomas (malignant tumors derived from striated muscle cells), among others.

Carcinomas that can be treated or prevented with the compounds of the present invention include tumors arising from epithelial tissue, such as glands, breast, skin, and linings of the urogenital, digestive, and respiratory systems. Lung, cancer and prostate cancers can be treated or prevented. Breast cancers that can be treated or prevented with the compounds of the present invention include both invasive (e.g., infiltrating ductal carcinoma, infiltrating lobular carcinoma infiltrating ductal & lobular carcinoma, medullary carcinoma, mucinous (colloid) carcinoma, comedocarcinoma, paget's disease, papillary carcinoma, tubular carcinoma, adenocarcinoma (NOS) and carcinoma (NOS)) and non-invasive carcinomas (e.g., intraductal carcinoma, lobular carcinoma in situ (LCIS), intraductal & LCIS, papillary carcinoma, comedocarcinoma). The present invention can also be used to treat or prevent metastatic breast cancer. Non-limiting examples of metastatic breast cancer include bone, lung and liver cancer.

Prostate cancers that can be treated or prevented with the compounds of the present invention include localized, regional and metastatic prostate cancer. Localized prostate cancers include A1-A2, T1a-T1b, T1c, B0-B2 or T2a-T2c. C1-C2 or T3a-N0, prostate cancers extending beyond the prostate but without lymph node involvement, are also contemplated. Regional prostate cancers include D1 or N1-M0, while metastatic prostate cancers include D2 or M1. Metastatic prostate cancers include bone and brain cancers.

Other cancers that can be treated or prevented with the compounds of the present invention include, but are not limited to, cancers of the cancers include those of the bowel, bladder, brain, cervix, colon, rectum, esophagus, eye, head and neck, liver, kidney, larynx, lung, skin, ovary, pancreas, pituitary gland, stomach, testicles, thymus, thyroid, uterus, and vagina as well as adrenocortical cancer, carcinoid tumors, endocrine cancers, endometrial cancer, gastric cancer, gestational trophoblastic tumors, islet cell cancer, and mesothelioma.

Lymphomas that can be treated or prevented with the compounds of the serum cocktail include are tumors arising from the lymph or spleen. lymph nodes and spleen, causing excessive production of lymphocytes, including both Hodgkin's disease and Non-Non-Hodgkin's lymphoma. The term “Hodgkin's Disease” is intended to include diseases classified as such by the REAL and World Health Organization (WHO) classifications known to those of skill in the art, including classical Hodgkin's disease (i.e., nodular sclerosis, mixed cellularity, lymphocyte depletion or lymphocyte rich) or lymphocyte predominance Hodgkin's disease. The term “Non-Hodgkin's lymphoma” is used to refer 30 lymphomas classified by WHO (Harris N L, Jaffe E S, Kiebold J, Flandrin G, Muller-Hermelink H K, Vardiman J. Lymphoma classification—from controversy to consensus: the REAL and WHO Classification of lymphoid neoplasms. Ann Oncol. 2000;11(suppl 1):53-S10), including but not limited to:

B-cell non-Hodgkin's lymphomas such as small lymphocytic lymphoma (SLL/CLL), mantle cell lymphoma (MCL), follicular lymphoma marginal zone lymphoma (MZL), extranodal (MALT lymphoma), nodal (Monocytoid B-cell lymphoma), splenic, diffuse large cell lymphoma, burkitt's lymphoma and lymphoblastic lymphoma.

T-cell non-Hodgkin's lymphoma's such as lymphoblastic lymphomas, peripheral T-cell lymphoma. Hepatosplenic gamma-delta T-cell lymphoma, subcutaneous panniculitis-like lymphoma, angioimmunoblastic T-cell lymphoma (AILD), extranodal NK/T cell lymphoma, nasal type, intestinal T-cell lymphoma (+/−enteropathy associated) (EATL), adult T-cell leukemia/lymphoma (HTLV-1 associated), mycosis fungoides/Sezary syndrome, anaplastic large cell lymphoma (ALCL), including both primary cuteous and primary systemic types.

Leukemias that can be treated or prevented with the compounds of the present invention include but are not limited to myeloid and lymphocytic (sometimes referred to as B or T cell leukemias) or myeloid leukemias, both chronic and acute. The myeloid leukemias include chronic myeloid leukemia (CML) and acute myeloid leukemia (AML) (i.e., acute nonlymphocytic leukemia (ANLL)). The lymphocytic leukemias include acute lymphocytic leukemia (ALL), chronic lymphocytic leukemia (CLL)(i.e., chronic granulocytic leukemia) and hairy cell leukemia (HCL).

Sarcomas that can be treated or prevented with the compounds of the present invention include both bone and soft-tissue sarcomas of the muscles, tendons, fibrous tissues, fat, blood vessels nerves, and synovial tissues. Non-limiting examples include fibrosacromas, rhabdomyosarcomas, liposarcomas, synovial sarcomas, angiosacromas, neurofibrosarcomas, gastrointestinal stroma tumors, Kaposi's sacroma, Ewing's sarcoma, alveolar soft-part sarcoma, angiosarcoma, dermatofibrosarcoma protuberans, epithelioid sarcoma, extraskeletal chondrosarcoma, extraskeletal osteosarcoma, fibrosarcoma, leiomyosarcoma, liposarcoma, malignant fibrous histiocytoma, malignant hemangiopericytoma, malignant mesenchymoma, malignant schwannoma, malignant peripheral nerve sheath tumor, parosteal osteosarcoma, peripheral neuroectodermal tumors, rhabdomyosarcoma, synovial sarcoma, and sarcoma, NOS.

Diseases of abnormal cell proliferation other than cancer can be treated or prevented with the compounds of the present invention. Diseases association with the abnormal proliferation of vascular smooth muscle cells are included, including, for example, benign tumors. Non-limiting examples of benign tumors include benign bone, brain and liver tumors.

Other diseases associated with abnormal cell proliferation include, for example, atherosclerosis and restenosis. Diseases associated with abnormal proliferation of over-proliferation and accumulation of tissue mast cells are also included, such as cutaneous mastocytosis (CM) and Urticaria pigmentosa. Diseases associated with abnormal proliferation of xesangial cell proliferation are also contemplated, including but not limited to IgA nephropathy, membranoproliferative glomerulonephritis (GN), lupus nephritis and diabetic nephropathy.

The present invention can also be used to treat or prevent lymphangiomyomatosis (LAM), as well as other diseases associated with abnormal cell proliferation known to those skilled in the art.

### VI. COMBINATION AND ALTERNATION THERAPY

Any of the compounds disclosed herein can be administered in combination or alternation with one or more other biologically active agents to increase its effectiveness against the abnormal cell proliferation disorder. Furthermore, the compounds of the invention can be administered in combination or alternation with a second biologically active agent to increase its effectiveness against inflammatory diseases or autoimmune diseases.

In combination therapy, effective dosages of two or more agents are administered together, whereas during alternation therapy an effective dosage of each agent is administered serially. The dosages will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. In certain embodiments, the ratio of compound of the invention to addition agent ranges between 1:0.0001 and 1:100, in certain embodiments between 1:0.001 and 1:50 or 1:0.01 and 1:10.

The efficacy of a drug can be prolonged, augmented, or restored by administering the compound in combination or alternation with a second, and perhaps third, agent that induces a different biological pathway from that caused by the principle drug. Alternatively, the pharmacokinetics, biodistribution or other parameter of the drug can be altered by such combination or alternation therapy. In general, combination therapy is typically preferred over alternation therapy because it induces multiple simultaneous stresses on the condition.

Any method of alternation can be used that provides treatment to the patient. Nonlimiting examples of alternation patterns include 1-6 weeks of administration of an effective amount of one agent followed by 1-6 weeks of administration of an effective amount of a second agent. The alternation schedule can include periods of no treatment. Combination therapy generally includes the simultaneous administration of an effective ratio of dosages of two or more active agents.

Illustrative examples of specific agents that can be used in combination or alternation with the compounds of the present invention are described below in regard to asthma and arthritis. The agents set out below or others can alternatively be used to treat a host suffering from any of the other disorders listed in Section IV or that are mediated by LO, and preferably 15-LO.

### Asthma

In one embodiment, the compound of the present invention is administered in combination or alternation with heparin, frusemide, ranitidine, an agent that effects respiratory function, such as DNAase, or immunosuppressive agents, IV gamma globulin, troleandomycin, cyclosporin (Neoral), methotrexate, FK-506, gold compounds such as Myochrysine (gold sodium thiomalate), platelet activating factor (PAF) antagonists such as thromboxane inhibitors, leukotriene-D₄-receptor antagonists such as Accolate (zafirlukast), Ziflo (zileuton), leukotriene C₁ or C₂ antagonists and inhibitors of leukotriene synthesis such as zileuton for the treatment of asthma, or an inducible nitric oxide synthase inhibitor.

In another embodiment, the active compound is administered in combination or alternation with one or more other prophylactic agent(s). Examples of prophylactic agents that can be used in alternation or combination therapy include but are not limited to sodium cromoglycate, Intal (cromolyn sodium, Nasalcrom, Opticrom, Crolom, Ophthalmic Crolom), Tilade (nedocromil, nedocromil sodium) and ketotifen.

In another embodiment, the active compound is administered in combination or alternation with one or more other β₂-adrenergic agonist(s) (β-agonists). Examples of β₂-adrenergic agonists (β-agonists) that can be used in alternation or combination therapy include but are not limited to albuterol (salbutamol, Proventil, Ventolin), terbutaline, Maxair (pirbuterol), Serevent (salmeterol), epinephrine, metaproterenol (Alupent, Metaprel), Brethine (Bricanyl, Brethaire, terbutaline sulfate), Tornalate (bitolterol), isoprenaline, ipratropium bromide, bambuterol hydrochloride, bitolterol meslyate, broxaterol, carbuterol hydrochloride, clenbuterol hydrochloride, clorprenaline hydrochloride, efirmoterol fumarate, ephedra (source of alkaloids), ephedrine (ephedrine hydrochloride, ephedrine sulfate), etafedrine hydrochloride, ethylnoradrenaline hydrochloride, fenoterol hydrochloride, hexoprenaline hydrochloride, isoetharine hydrochloride, isoprenaline, mabuterol, methoxyphenamine hydrochloride, methylephedrine hydrochloride, orciprenaline sulphate, phenylephrine acid tartrate, phenylpropanolamine (phenylpropanolamine polistirex, phenylpropanolamine sulphate), pirbuterol acetate, procaterol hydrochloride, protokylol hydrochloride, psuedoephedrine (psuedoephedrine polixtirex, psuedoephedrine tannate, psuedoephedrine hydrochloride, psuedoephedrine sulphate), reproterol hydrochloride, rimiterol hydrobromide, ritodrine hydrochloride, salmeterol xinafoate, terbutaline sulphate, tretoquinol hydrate and tulobuterol hydrochloride.

In another embodiment, the active compound is administered in combination or alternation with one or more other corticosteriod(s). Examples of corticosteriods that can be used in alternation or combination therapy include but are not limited to glucocorticoids (GC), Aerobid (Aerobid-M, flunisolide), Azmacort (triamcinolone acetonide), Beclovet (Vanceril, beclomethasone dipropionate), Flovent (fluticasone), Pulmicort (budesonide), prednisolone, hydrocortisone, adrenaline, Aldlometasone Dipropionate, Aldosterone Amcinonide, Beclomethasone Dipropionate, Bendacort, Betamethasone (Betamethasone Acetate, Betamnethasone Benzoate, Betamethasone Dipropionate, Betamethasone Sodium Phosphate, Betamethasone Valerate), Budesonide, Ciclomethasone, Ciprocinonide, Clobetasol Propionate, Clobetasone Butyrate, Clocortolone Pivalate, Cloprednol, Cortisone Acetate, Cortivazol, Deflazacort, Deoxycortone Acetate (Deoxycortone Pivalate), Deprodone, Desonide, Desoxymethasone, Dexamethasone (Dexamethasone Acetate, Dexamethasone Isonicotinate, Dexamethasone Phosphate, Dexamethasone Sodium Metasulphobenzoate, Dexamethasone Sodium Phosphate), Dichlorisone Acetate, Diflorasone Diacetate, Diflucortolone Valerate, Difluprednate, Domoprednate, Endrysone, Fluazacort, Fluclorolone Acetonide, Fludrocortisone Acetate, Flumethasone (Flumethasone Pivalate), Flunisolide, Fluocinolone Acetonide, Fluocinonide, Fluocortin Butyl, Fluocortolone (Fluocortolone Hexanoate, Fluocortolone Pivalate), Fluorometholone (Fluorometholone Acetate), Fluprednidene Acetate, Fluprednisolone, Flurandrenolone, Fluticasone Propionate, Formocortal, Halcinonide, Halobetasol Propionate, Halometasone, Hydrocortamnate Hydrochloride, Hydrocortisone (Hydrocortisone Acetate, Hydrocortisone Butyrate, Hydrocortisone Cypionate, Hydrocortisone Hemisuccinate, Hydrocortisone Sodium Phosphate, Hydrocortisone Sodium Succinate, Hydrocortisone Valerate), Medrysone, Meprednisone, Methylprednisolone (Methylprednisolone Acetate, Methylprednisolone, Hemisuccinate, Methylprednisolone Sodium Succinate), Mometasone Furoate, Paramethasone Acetate, Prednicarbate, Prednisolamate Hydrochloride, Prednisolone (Prednisolone Acetate, Prednisolone Hemisuccinate, Prednisolone Hexanoate, Prednisolone Pivalate, Prednisolone Sodium Metasulphobenzoate, Prednisolone Sodium Phosphate, Prednisolone Sodium Succinate, Prednisolone Steaglate, Prednisolone Tebutate), Prednisone (Prednisone Acetate), Prednylidene, Procinonide, Rimexolone, Suprarenal Cortex, Tixocortol Pivalate, Triamcinolone (Triamcinolone Acetonide, Triamcinolone Diacetate and Triamcinolone Hexacetonide).

In another embodiment, the active compound is administered in combination or alternation with one or more other antihistamine(s) (H₁ receptor antagonists). Examples of antihistamines (H₁ receptor antagonists) that can be used in alternation or combination therapy include alkylamines, ethanolamines, ethylenediamines, piperazines, piperidines or phenothiazines. Some non-limiting examples of antihistamines are Chlortrimeton (Teldrin, chlorpheniramine), Atrohist (brompheniramine, Bromarest, Bromfed, Dimetane), Actidil (triprolidine), Dexchlor (Poladex, Polaramine, dexchlorpheniramine), Benadryl (diphen-hydramine), Tavist (clemastine), Dimetabs (dimenhydrinate, Dramamine, Marmine), PBZ (tripelennamine), pyrilamine, Marezine (cyclizine), Zyrtec (cetirizine), hydroxyzine, Antivert (meclizine, Bonine), Allegra (fexofenadine), Hismanal (astemizole), Claritin (loratadine), Seldane (terfenadine), Periactin (cyproheptadine), Nolamine (phenindamine, Nolahist), Phenameth (promethazine, Phenergan), Tacaryl (methdilazine) and Temaril (trimeprazine).

Alternatively, the compound of the present invention is administered in combination or alternation with (a) xanthines and methylxanthines, such as Theo-24 (theophylline, Slo-Phylline, Uniphyllin, Slobid, Theo-Dur), Choledyl (oxitriphylline), aminophylline; (b) anticholinergic agents (antimuscarinic agents) such as belladonna alkaloids, Atrovent (ipratropium bromide), atropine, oxitropium bromide; (c) phosphodiesterase inhibitors such as zardaverine; (d) calcium antagonists such as nifedipine; or (e) potassium activators such as cromakalim for the treatment of asthma.

### Arthritic Disorders

Rheumatoid arthritis is one of the most common of the autoimmune diseases. Current treatments utilize three general classes of drugs (Schumacher, H. R. ed., Pimer on the Rheumatic Diseases, Ninth edition, Arthritis Foundation, Atlanta, Ga. (1988): anti-inflammatory agents (aspirin, non-steroidal anti-inflammatory drugs and low dose corticosteriods); disease-modifying antirheumatic drugs, known as “DMARDs” (anti-malarials, gold salts, penicillamine, and sulfasalazine) and immunosuppressive agents (azathioprine, chlorambucil, high dose corticosteroids, cyclophosphamide, methotrexate, nitrogen mustard, 6-mercaptopurine, vincristine, hydroxyurea, and cyclosporin A). None of the available drugs are completely effective, and most are limited by severe toxicity.

When used to treat rheumatoid arthritis, the compounds of the present invention can be used in alternation or combination with any agent or drug known for the treatment of rheumatoid arthritic, including but not limited to: Remicade® (infliximab); methotrexate; Nonsteroidal anti-inflammatory drugs (NSAIDs) such as aspirin and ibuprofen; corticosteroid medications, such as Prednisone; Leflunomide; biologic agents such as etanercept, infliximab, adalimumab, and anakinra; celecoxib; tetracyclines; tumour necrosis factor (TNF) antagonists; nonsteroidal anti-inflammatories; cyclooxygenase-2 inhibitors; interleukin-1-receptor antagonist

Drugs in clinical investigation are contemplated, including but not limited to: 681323 (p38 alpha kinase inhibitor) (GlaxoSmithKline); 683699 (T-0047) (dual alpha 4 integrin antaginist) (GlaxoSmithKline); ABT-963 (Abbott Laboratories); AGIX-4207 (Atherogenics); alpha-L-iduronidase (Genzyme General), AMG719 (Amgen); AnergiX.RA (Corixa); anti-CD11 humanized MAb (Genentech); Arava (Aventis Pharmaceuticals); CDP 870 (Pfizer); CDP-870 (Pfizer); Celebrex (Pfizer); COX 189 (Novartis); eculizumab (Alexion Pharmaceuticals); HuMax-IL15 (Amgen); IDEC 151 (IDEC Pharmaceuticals); IDEC-151/clenoliximab (IDEC Pharmaceuticals; IL-1 trap (Rengeneron Pharmaceuticals); interleukin-1 (Regeneron Pharmaceuticals); interleukin-18 (Regeneron Pharmaceuticals); J695 (Abbott Laboratories); Oraprine (DORBioPharma); pegsunercept (soluble tumor necrosis factor-a receptor type 1)(Amgen); pralnacasan (Aventis); Prograf (Fujisawa Healthcare); r-IL-18 by (Serono); R1487 (kinase inhibitor)(Roche); Rituxan (Genentech); SB281832 (GlaxoSmithKline); SCIO-323 (Scio); SCIO-469 (Scio) and Vitaxin (MedImmune).

In one embodiment, the compound of the present invention can also be administered in combination or alternation with apazone, amitriptyline, chymopapain, collegenase, cyclobenzaprine, diazepam, fluoxetine, pyridoxine, ademetionine, diacerein, glucosamine, hylan (hyaluronate), misoprostol, paracetamol, superoxide dismutase mimics, TNFα receptor antagonists, TNFα antibodies, P38 Kinase inhibitors, tricyclic antidepressents, cJun kinase inhibitors or immunosuppressive agents, IV gamma globulin, troleandomycin, cyclosporin (Neoral), methotrexate, FK-506, gold compounds such as Myochrysine (gold sodium thiomalate), platelet activating factor (PAF) antagonists such as thromboxane inhibitors, leukotriene-D₄-receptor antagonists such as Accolate (zafirlukast), Ziflo (zileuton), leukotriene C₁, C₂ antagonists and inhibitors of leukotriene synthesis such as zileuton for the treatment of arthritic disorders, inducible nitric oxide sythase inhibitors.

In another embodiment, the active compound is administered in combination or alternation with one or more other corticosteriod(s). Examples of corticosteriods that can be used in alternation or combination therapy include but are not limited to glucocorticoids (GC), Aerobid (Aerobid-M, flunisolide), Azmacort (triamcinolone acetonide), Beclovet (Vanceril, beclomethasone dipropionate), Flovent (fluticasone), Pulmicort (budesonide), prednisolone, hydrocortisone, adrenaline, Alclometasone Dipropionate, Aldosterone, Amcinonide, Beclomethasone Dipropionate, Bendacort, Betamethasone (Betamethasone Acetate, Betamethasone Benzoate, Betamethasone Dipropionate, Betamethasone Sodium Phosphate, Betamethasone Valerate), Budesonide, Ciclomethasone, Ciprocinonide, Clobetasol Propionate, Clobetasone Butyrate, Clocortolone Pivalate, Cloprednol, Cortisone Acetate, Cortivazol, Deflazacort, Deoxycortone Acetate (Deoxycortone Pivalate), Deprodone, Desonide, Desoxymethasone, Dexamethasone (Dexamethasone Acetate, Dexamethasone Isonicotinate, Dexamethasone Phosphate, Dexamnethasone Sodium Metasulphobenzoate, Dexamethasone Sodium Phosphate), Dichlorisone Acetate, Diflorasone Diacetate, Diflucortolone Valerate, Difluprednate, Domoprednate, Endrysone, Fluazacort, Fluclorolone Acetonide, Fludrocortisone Acetate, Flumethasone (Flumethasone Pivalate), Flunisolide, Fluocinolone Acetonide, Fluocinonide, Fluocortin Butyl, Fluocortolone (Fluocortolone Hexanoate, Fluocortolone Pivalate), Fluorometholone (Fluorometholone Acetate), Fluprednidene Acetate, Fluprednisolone, Flurandrenolone, Fluticasone Propionate, Formocortal, Halcinonide, Halobetasol Propionate, Halometasone, Hydrocortamate Hydrochloride, Hydrocortisone (Hydrocortisone Acetate, Hydrocortisone Butyrate, Hydrocortisone Cypionate, Hydrocortisone Hemisuccinate, Hydrocortisone Sodium Phosphate, Hydrocortisone Sodium Succinate, Hydrocortisone Valerate), Medrysone, Meprednisone, Methylprednisolone (Methylprednisolone Acetate, Methylprednisolone, Hemisuccinate, Methylprednisolone Sodium Succinate), Mometasone Furoate, Paramethasone Acetate, Prednicarbate, Prednisolamate Hydrochloride, Prednisolone (Prednisolone Acetate, Prednisolone Hemisuccinate, Prednisolone Hexanoate, Prednisolone Pivalate, Prednisolone Sodium Metasulphobenzoate, Prednisolone Sodium Phosphate, Prednisolone Sodium Succinate, Prednisolone Steaglate, Prednisolone Tebutate), Prednisone (Prednisone Acetate), Prednylidene, Procinonide, Rimexolone, Suprarenal Cortex, Tixocortol Pivalate, Triamcinolone (Triamcinolone Acetonide, Triamcinolone Diacetate and Triamcinolone Hexacetonide).

In another embodiment, the active compound is administered in combination or alternation with one or more other non-steroidal anti-inflammatory drug(s) (NSAIDS). Examples of NSAIDS that can be used in alternation or combination therapy are carboxylic acids, propionic acids, fenamates, acetic acids, pyrazolones, oxicans, alkanones, gold compounds and others that inhibit prostaglandin synthesis, preferably by selectively inhibiting cylcooxygenase-2 (COX-2). Some nonlimiting examples of COX-2 inhibitors are Celebrex (celecoxib) and Vioxx (rofacoxib). Some non-limiting examples of NSAIDS are aspirin (acetylsalicylic acid), Dolobid (diflunisal), Disalcid (salsalate, salicylsalicylate), Trisilate (choline magnesium trisalicylate), sodium salicylate, Cuprimine (penicillamine), Tolectin (tolmetin), ibuprofen (Motrin, Advil, Nuprin Rufen), Naprosyn (naproxen, Anaprox, naproxen sodium), Nalfon (fenoprofen), Orudis (ketoprofen), Ansaid (flurbiprofen), Daypro (oxaprozin), meclofenamate (meclofanamic acid, Meclomen), mefenamic acid, Indocin (indomethacin), Clinoril (sulindac), tolmetin, Voltaren (diclofenac), Lodine (etodolac), ketorolac, Butazolidin (phenylbutazone), Tandearil (oxyphenbutazone), piroxicam (Feldene), Relafen (nabumetone), Myochrysine (gold sodium thiomalate), Ridaura (auranofin), Solganal (aurothioglucose), acetaminophen, colchicine, Zyloprim (allopurinol), Benemid (probenecid), Anturane (sufinpyrizone), Plaquenil (hydroxychloroquine), Aceclofenac, Acemetacin, Acetanilide, Actarit, Alclofenac, Alminoprofen, Aloxiprin, Aluminium Aspirin, Amfenac Sodium, Amidopyrine, Aminopropylone, Ammonium Salicylate, Ampiroxicam, Amyl Salicylate, Anirolac, Aspirin, Auranofin, Aurothioglucose, Aurotioprol, Azapropazone, Bendazac (Bendazac Lysine), Benorylate, Benoxaprofen, Benzpiperylone, Benzydamine hydrochloride, Bomyl Salicylate, Bromfenac Sodium, Bufexamac, Bumadizone Calcium, Butibufen Sodium, Capsaicin, Carbaspirin Calcium, Carprofen, Chlorthenoxazin, Choline Magnesium Trisalicylate, Choline Salicylate, Cinmetacin, Clofexamide, Clofezone, Clometacin, Clonixin, Cloracetadol, Cymene, Diacerein, Diclofenac (Diclofenac Diethylammonium Salt, Diclofenac Potassium, Diclofenac Sodium), Diethylamine Salicylate, Diethylsalicylamide, Difenpiramide, Diflunisal, Dipyrone, Droxicam, Epirizole, Etenzamide, Etersalate, Ethyl Salicylate, Etodolac, Etofenamate, Felbinac, Fenbufen, Fenclofenac, Fenoprofen Calcium, Fentiazac, Fepradinol, Feprazone, Floctafenine, Flufenamic, Flunoxaprofen, Flurbiprofen (Flurbiprofen Sodium), Fosfosal, Furprofen, Glafenine, Glucametacin, Glycol Salicylate, Gold Keratinate, Harpagophytum Procumbens, Ibufenac, Ibuprofen, Ibuproxam Imidazole Salicylate, Indomethacin (Indomethacin Sodium), Indoprofen, Isamifazone, Isonixin, Isoxicam, Kebuzone, Ketoprofen, Ketorolac Trometamol, Lithium Salicylate, Lonazolac Calcium, Lomoxicam, Loxoprofen Sodium, Lysine Aspirin, Magnesium Salicylate, Meclofenamae Sodium, Mefenamic Acid, Meloxicam, Methyl Butetisalicylate, Methyl Gentisate, Methyl Salicylate, Metiazinic Acid, Metifenazone, Mofebutazone, Mofezolac, Morazone Hydrochloride, Morniflumate, Morpholine Salicylate, Nabumetone, Naproxen (Naproxen Sodium), Nifenazone, Niflumic Acid, Nimesulide, Oxametacin, Oxaprozin, Oxindanac, Oxyphenbutazone, Parsalmide, Phenybutazone, Phenyramidol Hydrochloride, Picenadol Hydrochloride, Picolamine Salicylate, Piketoprofen, Pirazolac, Piroxicam, Pirprofen, Pranoprofen, Pranosal, Proglumetacin Maleate, Proquazone, Protizinic Acid, Ramifenazone, Salacetamide, Salamidacetic Acid, Salicylamide, Salix, Salol, Salsalate, Sodium Aurothiomalate, Sodium Gentisate, Sodium Salicylate, Sodium Thiosalicylate, Sulindac, Superoxide Dismutase (Orgotein, Pegorgotein, Sudismase), Suprofen, Suxibuzone, Tenidap Sodium, Tenoxicam, Tetrydamine, Thurfyl Salicylate, Tiaprofenic, Tiaramide Hydrochloride, Tinoridine Hydrochloride, Tolfenamic Acid, Tometin Sodium, Triethanolamine Salicylate, Ufenamate, Zaltoprofen, Zidometacin and Zomepirac Sodium.

### Transplant Rejection

In addition to their use in treating autoimmune conditions, immunosuppressive agents have also been used in treating or preventing transplantation rejection. Organ transplantation involving human organ donors and human recipients (allografts), and non-human primate donors and human recipients (xenografts), has received considerable medical and scientific attention (Roberts, J. P., et al., Ann. Rev. Med., 40:287 (1989); Platt, J. L., et al., Immunol. Today 11(2):450 (1990); Keown, P. A., Ann. Rev. Trans., Clin. Transplants 205-223, (1991). To a great extent, these efforts has been aimed at eliminating, or at least reducing, the problem of rejection of the transplanted organ. In the absence of adequate immunosuppressive therapy, the transplanted organ is destroyed by the host immune system. Presently, the most commonly used agents for preventing transplant rejection include corticosteriods, antimetabolite drugs that reduce lymphocyte proliferation by inhibiting DNA and RNA synthesis such as azathioprine, immunosuppressive drugs such as cyclosporin A, which specifically inhibits T cell activation, and specific antibodies directed against T lymphocytes or surface receptors that mediate their activation (Briggs J. D., Immunology Letters July 29(1-2):89-94 (1991). All of these drug therapies are limited in effectiveness, in part because the doses needed for effective treatment of transplant rejection may increase the patient's susceptibility to infection by a variety of opportunistic invaders, and in part because of direct toxicity and other side effects. For example, cyclosporin A, currently the most commonly used agent, is significantly toxic to the kidney. This nephrotoxicity limits the quantity of drug that can be safely given.

### Antiproliferative Agents

In one embodiment, the compound(s) of the present invention can be administered in combination or alternation one or more anti-proliferative agents. Any of the antiproliferative agents listed below, or any other such agent known or discovered to exhibit an antiproliferative effect can be used in combination or alternation with the present invention to achieve a combination therapeutic effect.

Representative adjuncts include levamisole, gallium nitrate, granisetron, sargramostim strontium-89 chloride, filgrastim, pilocarpine, dexrazoxane, and ondansetron. Physicians' Desk Reference, 50th Edition, 1996.

Representative androgen inhibitors include flutamide and leuprolide acetate. Physicians' Desk Reference, 50th Edition, 1996.

Representative antibiotic derivatives include doxorubicin, bleomycin sulfate, daunorubicin, dactinomycin, and idarubicin.

Representative antiestrogens include tamoxifen citrate and analogs thereof. Physicians' Desk Reference, 50th Edition, 1996. Additional antiestrogens include nonsteroidal antiestrogens such as toremifene, droloxifene and roloxifene. Magarian et al., Current Medicinal Chemistry, 1994, Vol. 1, No. 1.

Representative antimetabolites include fluorouracil, fludarabine phosphate, floxuridine, interferon alfa-2b recombinant, methotrexate sodium, plicamycin, mercaptopurine, and thioguanine. Physicians' Desk Reference, 50th Edition, 1996.

Representative cytotoxic agents include doxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci. Physicians' Desk Reference, 50th Edition, 1996.

Representative hormones include medroxyprogesterone acetate, estradiol, megestrol acetate, octreotide acetate, diethylstilbestrol diphosphate, testolactone, and goserelin acetate. Physicians' Desk Reference, 50th Edition, 1996.

Representative immunodilators include aldesleukin. Physicians' Desk Reference, 50th Edition, 1996.

Representative nitrogen mustard derivatives include melphalan, chlorambucil, mechlorethamine, and thiotepa. Physicians' Desk Reference, 50th Edition, 1996.

Representative steroids include betamethasone sodium phosphate and betamethasone acetate. Physicians' Desk Reference, 50th Edition, 1996.

Representative antineoplastic agents include paclitaxel or doxorubicin.

Additional suitable chemotherapeutic agents include alkylating agents, antimitotic agents, plant alkaloids, biologicals, topoisomerase I inhibitors, topoisomerase II inhibitors, and synthetics. AntiCancer Agents by Mechanism, tttp://www.dtp.nci.nih.gov/docs/cancer/searches/standard_mechanism_list.html, Apr. 12, 1999; Approved Anti-Cancer Agents, http://www.ctep.info.nih.gov/handbook/HandBookText/fda_agen.htm, pages 1-7, Jun. 18, 1999; MCMP 611 Chemotherapeutic Drugs to Know, http//www.vet.purdue.edu/depts/bms/courses/mcmp611/chrx/drg2no61.html, Jun. 24, 1999; and Chemotherapy, http://www.vetmed.lsu.edu/oncology/Chemotherapy.htm, Apr. 12, 1999.

Representative alkylating agents include asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864. AntiCancer Agents by Mechanism, http://dtp.nci.nih.gov/docs/cancer/searches/standardmechanism_list.html,

Apr. 12, 1999.

Representative antimitotic agents include allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate. AntiCancer Agents by Mechanism, http://dtp.nci.nih.gov/docs/cancer/searches/standard_mechanism_list.html, Apr. 12, 1999.

Representative plant alkaloids include actinomycin D, bleomycin, L-asparaginase, idarubicin, vinblastine sulfate, vincristine sulfate, mitramycin, mitomycin, daunorubicin, VP-16-213, VM-26, navelbine and taxotere. Approved Anti-Cancer Agents, http://ctep.info.nih.gov/handbook/HandBookText/fda_agent.htm, Jun. 18, 1999.

Representative biologicals include alpha interferon, BCG, G-CSF, GM-CSF, and interleukin-2. Approved Anti-Cancer Agents, http://ctep.info.nih.gov/handbook/HandBookText/fda_agent.htm, Jun. 18, 1999.

Representative topoisomerase I inhibitors include camptothecin, camptothecin derivatives, and morpholinodoxorubicin. AntiCancer Agents by Mechanism, http://dtp.nci.nih.gov/docs/cancer/searches/standard_mechanism_list.html, Apr. 12, 1999.

Representative topoisomerase II inhibitors include mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM- and 26 and VP-16. AntiCancer Agents by Mechanism, http://dtp.nci.nih.gov/docs/cancer/searches/standard_mechanism_list.html, Apr. 12, 1999.

Representative synthetics include hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium. Approved Anti-Cancer Agents, http://ctep.info.nih.gov/handbook/HandBookText/fda_agen.htm, Jun. 18, 1999.

Representative antibodies include Monoclonal antibodies directed to proliferating cells such as Rituximab (anti-CD20) for B-cell tumors and herceptin.

Drugs in clinical trials for cancer are specifically contemplated including, but not limited to: 715992 (kinesin inhibitor)(GlaxoSmithKline); Advexin (Introgen Therapeutics); AG-002037 (Pfizer); APC8024 (Dendreon); atrasentan (ABT-627); BIBH 1 (Boerhinger-Ingelheim) CCI 779 (Wyeth Pharmaceuticals); CEA Vac (Titan Pharmaceuticals); CEA-CIDE (Immunomedics) CEA-Scan (Immunomedics); Celebrex (Pharmacia); CP-547, 632 (anti-VEGF tyrosine kinase)(OSI Pharmaceuticals); CP-724-714 (anti-ErbB2[HER-2 neu] tyrosine kinase)(OSI Pharmaceuticals); CpG 7909 (Aventis Pharmaceuticals); dendritic/cancer cell fusion (Genzyme Molecular Oncology); ERA 923 (tissue-selective estrogen receptor modulator-SERM) (Ligand Pharmaceuticals); Ethyol (MedImmune Oncology); fowlpox-(6D)-TRICOM/vaccinia-(6D)-TRICOM vaccine (National Cancer Institute); G-3139 (Genta); Gemzar (Eli Lilly); Genasense (Genta); GeneVax (Centocor); GPI-0100 immune enhancer (adjuvant)(Galencia Pharmaceuticals); GTI 2040 (Lorus Therapeutics); GTI 2501 (Lorus Therapeutics); H11 (Viventia Biotech); interleukin-4 (IL-4) (National Cancer Institute); irofulven (National Cancer Institute); liquid IL-2 (Chiron); MAb antibody 3A1 (National Cancer Institute); multitargeted antifolate I (Eli Lily); Myocet (Liposome Company); oral paclitaxel (IVAX Pharmaceuticals); P53 and RAS vaccine (National Cancer Institute); PD-183805 (Pfizer); Proleukin (Chiron); ProMune (Chiron); R1550 (Antisoma); RAS peptides (National Cancer Institute); rebeccamycin analog (National Cancer Institute); recombinant human chorionic gonadotropin (r-hCG) (Serono); RSR-13 (Allos Therapeutics); RSR-13 (Eli Lilly); Targretin (Ligand Pharmaceuticals); tariquidar (QLT); Taxotere (Aventis Pharmaceuticals); TLK286 (Telik); vaccina-MUC-1 vaccine (Therion Biologics); vaccinia-MUC-1 vaccine (National Cancer Institute); Xtotax (Cell Therapeutics); Xyotax (Cell Therapeutics); Yondelis (ET-743)(Johnson & Johnson); Zarnestra (Johnson & Johnson); ZD6126 and ZD6474 (AstraZeneca); and Zoladex (AstraZeneca).

### Gene Therapy

In another embodiment, the compounds of the present invention can also be used in combination or alternation with gene therapy for the treatment or prevention of abnormal cell proliferation, including cancer.

Eukaryotic cells that may be transduced with vectors (i.e., infectious viral particles or plasmids) containing a gene therapeutic, but are not limited to, primary cells, such as primary nucleated blood cells, such as leukocytes, granulocytes, monocytes, macrophages, lymphocytes (including T-lymphocytes and B-lymphocytes), totipotent stem cells, and tumor infiltrating lymphocytes (TIL cells); bone marrow cells; endothelial cells; epithelial cells; keratinocytes; stem cells; hepatocytes, including hepatocyte precursor cells; hepatocytes, including hepatocyte precursor cells; fibroblasts; mesenchymal cells; mesothelial cells; parenchymal cells, or other cells of tumor derivation.

Optionally, the vector can also contain genes that enhance the therapeutic effects of the cell. Examples of suitable genes include those that encode cytokines such as TNF, GMCSF, interleukins (interleukins 1-18), interferons (alpha, beta, gamma-interferons).

In general, a gene cannot be directly inserted into a cell. It must be delivered to the cell using a carrier known as a “vector.” The most common types of vectors used in gene therapy are viruses. Scientists use viruses because they have a unique ability to attach to or enter a cell's DNA. Viruses used as vectors in gene therapy are genetically disabled; they are unable to reproduce themselves, though they can replicate coordinately with the cellular DNA. Many gene therapy clinical trials rely on mouse retroviruses to deliver the desired gene. Other viruses used as vectors include adenoviruses, adeno-associated viruses, poxviruses and the herpes virus.

Viral vectors all induce some degree of immunological response and may have other safety risks, such as insertional mutagenesis and direct toxicity. Furthermore, large-scale production may be difficult to achieve. Therefore, in some embodiments of the invention, non-viral methods of gene transfer are used in combination or alternation with the heterologous plasma or serum cocktail. These non-viral vectors may require only a small number of proteins, have a virtually infinite capacity, have no infectious or mutagenic capability, and large-scale production is possible using pharmaceutical techniques. There are at least three methods of non-viral DNA transfer, including naked DNA, liposomes and molecular conjugates.

### ImmunoTherapy

In yet another embodiment, the compounds of the present invention can be administered in combination or alternation with any of the immunotherapy agents or drugs. Any of the immunotherapy agents listed below, or any other such agent known or discovered to exhibit an immunotherapeutic effect can be used in combination or alternation with the present invention to achieve a combination therapeutic effect including: cytokines such as interferon-alpha, interferon-beta, interferon-gamma, and tumor necrosis factor; monoclonal antibodies such as Rituximab (Rituxan) and trastuzumab (Herceptin); bone and marrow stem cell transplants, including twin-donors, allogenic-donors and mismatched-donors, and more particularly including non-ablative allogeneic stem cell transplantation and autologous transplantation; immunotoxins, hybrid proteins consisting of an antibody and a toxin; cancer vaccines including dendritic cell cancer vaccines.

The composition of the present invention can also be used in combination or alternation with radiation therapy, in all forms known those skilled in the art.

When used to treat leukemia, the compounds of the present invention can be used in combination or alternation with tyrosine kinases such as Imatinib mesylate (Gleevec™) or other drugs known in the art for the treatment of leukemia.

### Atherosclerosis Agents

When used to treat atherosclerosis or restenosis, the compounds of the present invention can be used in alternation or combination with any agent or drug known for the treatment of either disease, including but not limited to: HMG-CoA reductase inhibitors including Pravastatin (Pravachol), Simvastatin (Zocor), Lovastatin (Mevacor, Altocor), Fluvastatin (Lescol), Atorvastatin (Lipitor), Rosuvastatin (Crestor); Fibric acid derivatives including Fenofibrate (Tricor) and Gemfibrozil (Lopid); Bile acid sequestrants including Cholestyramine (Questran, LoCholest, Prevalite) and Colestipol (Colestid); Antioxidants including vitamins C, E (Vita-Plus E, Softgels, Aquasol E), beta-carotene; Nicotinic acid derivatives including Niacin (Niaspan, Niacor, Slo-Niacin); other agents including but not limited to probucol, statins, aspirin, macrolide therapy, angiotensin-converting enzyme inhibitors, ACAT inhibitors, beta-blockers, atorvastatin, ticlopidine, and clopidogrel (inhibitors of platelet clumping) or other anticoagulants.

Drugs in clinical development for athersclerosis are also contemplated, including but not limited to AGI-1067 (Atherogenics), AGO-1067 (Atherogenics), Antrin (Pharmacyclics), avasimibe (ACAT inhibitor)(Pfizer), BO-653 (Chugai Pharmaceuticals), CETi-1 vaccine (AVANT Immunotherapuetics), CP-529,414 (Pfizer), SB480848 (Lp-PLA2 inhibitor) (GlaxoSmithKline), and Zithromax (Pfizer). Other clinical agents include the immunomodulatory drug DiNAC, N,N′-diacetyl-L-cystine (Astrazeneca); PPARgamma agonists (Abbott Laboratories);

### Psoriasis Agents

When used to treat psoriasis, the compounds of the present invention can be used in alternation or combination with any agent or drug known for the treatment of psoriasis, including but not limited to: tar (e.g., Exorex), topical corticosteroids, topical calcipotriene (Dovonex), topical tazarotene (Tazorac), anthralin (short contact therapy), corticosteroid tape (Cordran tape), and intralesional triamcinolone; UVB phototherapy; Psoralen+UVA (PUVA) and PUVA+acitretin (Re—PUVA); Acitretin (Soriatane); Methotrexate; Cyclosporine (Neoral); other immune inhibitors such as Mycophenolate mofetil, Hydroxyurea, and Leflunomide; other treatments include: Alefacept (AMEVIVE or LFA#TIP, Biogen); Oral retinoids; Cyclosporine; Etanercept and infliximab; topical vitamin D(3) analogues; dithranol

Drugs in clinical investigation are also contemplated, included but not limited to: Amevive (Biogen); BIRB 796 (Boehringer-Ingelheim Pharmaceuticals); Embrel (Amgen); Hectorol (Bone Care International); IDEC-114 (IDEC Pharmaceuticals); LFA-1 inhibitor (Biogen); ONTAK (Ligand Pharmaceuticals); PsorBan (CGC1072)(CellGate); r-IL-18 by (Serono); and Targretin Gel (Ligand Pharmaceuticals).

In another embodiment, the active compound is administered in combination or alternation with one or more other β₂-adrenergic agonist(s) (β agonists). Examples of β₂-adrenergic agonists (β agonists) that can be used in alternation or combination therapy include but are not limited to albuterol (salbutamol, Proventil, Ventolin), terbutaline, Maxair (pirbuterol), Serevent (salmeterol), epinephrine, metaproterenol (Alupent, Metaprel), Brethine (Bricanyl, Brethaire, terbutaline sulfate), Tornalate (bitolterol), isoprenaline, ipratropium bromide, bambuterol hydrochloride, bitolterol meslyate, broxaterol, carbuterol hydrochloride, clenbuterol hydrochloride, clorprenaline hydrochloride, efirmoterol fumarate, ephedra (source of alkaloids), ephedrine (ephedrine hydrochloride, ephedrine sulfate), etafedrine hydrochloride, ethylnoradrenaline hydrochloride, fenoterol hydrochloride, hexoprenaline hydrochloride, isoetharine hydrochloride, isoprenaline, mabuterol, methoxyphenamine hydrochloride, methylephedrine hydrochloride, orciprenaline sulphate, phenylephrine acid tartrate, phenylpropanolamine (phenylpropanolamine polistirex, phenylpropanolamine sulphate), pirbuterol acetate, procaterol hydrochloride, protokylol hydrochloride, psuedoephedrine (psuedoephedrine polixtirex, psuedoephedrine tannate, psuedoephedrine hydrochloride, psuedoephedrine sulphate), reproterol hydrochloride, rimiterol hydrobromide, ritodrine hydrochloride, salmeterol xinafoate, terbutaline sulphate, tretoquinol hydrate and tulobuterol hydrochloride.

In another embodiment, the active compound is administered in combination or alternation with one or more other corticosteriod(s). Examples of corticosteriods that can be used in alternation or combination therapy include but are not limited to glucocorticoids (GC), Aerobid (Aerobid-M, flunisolide), Azmacort (triamcinolone acetonide), Beclovet (Vanceril, beclomethasone dipropionate), Flovent (fluticasone), Pulmicort (budesonide), prednisolone, hydrocortisone, adrenaline, Alclometasone Dipropionate, Aldosterone, Amcinonide, Beclomethasone Dipropionate, Bendacort, Betamethasone (Betamethasone Acetate, Betamethasone Benzoate, Betamethasone Dipropionate, Betamethasone Sodium Phosphate, Betamethasone Valerate), Budesonide, Ciclomethasone, Ciprocinonide, Clobetasol Propionate, Clobetasone Butyrate, Clocortolone Pivalate, Cloprednol, Cortisone Acetate, Cortivazol, Deflazacort, Deoxycortone Acetate (Deoxycortone Pivalate), Deprodone, Desonide, Desoxymethasone, Dexamethasone (Dexamethasone Acetate, Dexamethasone Isonicotinate, Dexamethasone Phosphate, Dexamethasone Sodium Metasulphobenzoate, Dexamethasone Sodium Phosphate), Dichlorisone Acetate, Diflorasone Diacetate, Diflucortolone Valerate, Difluprednate, Domoprednate, Endrysone, Fluazacort, Fluclorolone Acetonide, Fludrocortisone Acetate, Flumethasone (Flumethasone Pivalate), Flunisolide, Fluocinolone Acetonide, Fluocinonide, Fluocortin Butyl, Fluocortolone (Fluocortolone Hexanoate, Fluocortolone Pivalate), Fluorometholone (Fluorometholone Acetate), Fluprednidene Acetate, Fluprednisolone, Flurandrenolone, Fluticasone Propionate, Formocortal, Halcinonide, Halobetasol Propionate, Halometasone, Hydrocortamate Hydrochloride, Hydrocortisone (Hydrocortisone Acetate, Hydrocortisone Butyrate, Hydrocortisone Cypionate, Hydrocortisone Hemisuccinate, Hydrocortisone Sodium Phosphate, Hydrocortisone Sodium Succinate, Hydrocortisone Valerate), Medrysone, Meprednisone, Methylprednisolone (Methylprednisolone Acetate, Methylprednisolone, Hemisuccinate, Methylprednisolone Sodium Succinate), Mometasone Furoate, Paramethasone Acetate, Prednicarbate, Prednisolamate Hydrochloride, Prednisolone (Prednisolone Acetate, Prednisolone Hemisuccinate, Prednisolone Hexanoate, Prednisolone Pivalate, Prednisolone Sodium Metasulphobenzoate, Prednisolone Sodium Phosphate, Prednisolone Sodium Succinate, Prednisolone Steaglate, Prednisolone Tebutate), Prednisone (Prednisone Acetate), Prednylidene, Procinonide, Rimexolone, Suprarenal Cortex, Tixocortol Pivalate, Triamcinolone (Triamcinolone Acetonide, Triamcinolone Diacetate and Triamcinolone Hexacetonide).

In another embodiment, the active compound is administered in combination or alternation with one or more other antihistamine(s) (H₁ receptor antagonists). Examples of antihistamines (H₁ receptor antagonists) that can be used in alternation or combination therapy include alkylamines, ethanolamines, ethylenediamines, piperazines, piperidines or phenothiazines. Some non-limiting examples of antihistamines are Chlortrimeton (Teldrin, chlorpheniramine), Atrohist (brompheniramine, Bromarest, Bromfed, Dimetane), Actidil (triprolidine), Dexchlor (Poladex, Polaramine, dexchlorpheniramine), Benadryl (diphen-hydramine), Tavist (clemastine), Dimetabs (dimenhydrinate, Dramamine, Marmine), PBZ (tripelennamine), pyrilamine, Marezine (cyclizine), Zyrtec (cetirizine), hydroxyzine, Antivert (meclizine, Bonine), Allegra (fexofenadine), Hismanal (astemizole), Claritin (loratadine), Seldane (terfenadine), Periactin (cyproheptadine), Nolamine (phenindamine, Nolahist), Phenameth (promethazine, Phenergan), Tacaryl (methdilazine) and Temaril (trimeprazine).

Alternatively, the compound of the present invention is administered in combination or alternation with
(a) xanthines and methylxanthines, such as Theo-24 (theophylline, Slo-Phylline, Uniphyllin, Slobid, Theo-Dur), Choledyl (oxitriphylline), aminophylline;
(b) anticholinergic agents (antimuscarinic agents) such as belladonna alkaloids, Atrovent (ipratropium bromide), atropine, oxitropium bromide;
(c) phosphodiesterase inhibitors such as zardaverine;
(d) calcium antagonists such as nifedipine; or
(e) potassium activators such as cromakalim for the treatment of asthma.

### VII. PHARMACEUTICAL COMPOSITIONS

The described compounds can be formulated as pharmaceutical compositions and administered for any of the disorders described herein, including autoimmune and inflammatory disorders and disorders of abnormal cell proliferation, in a host, including a human, in any of a variety of forms adapted to the chosen route of administration, including systemically, such as orally, or parenterally, by intravenous, intramuscular, topical, transdermal or subcutaneous routes.

The compound can be included in the pharmaceutically acceptable carrier or diluent in an amount sufficient to deliver to a patient a therapeutically effective amount of compound to treat autoimmune or anti-inflammatory disorders or disorders of abnormal cell proliferation or the symptoms thereof in vivo without causing serious toxic effects in the patient treated.

A typical dose of the derivatives for all of the above-mentioned conditions will be in the range from about 1 to 75 mg/kg, preferably 1 to 20 mg/kg, of body weight per day, more generally 0.1 to about 100 mg per kilogram body weight of the recipient per day. The effective dosage range of the prodrug can be calculated based on the weight of the parent derivative to be delivered.

The compounds are conveniently administered in units of any suitable dosage form, including but not limited to one containing 7 to 3000 mg, preferably 70 to 1400 mg of active ingredient per unit dosage form. An oral dosage of 50-1000 mg is usually convenient, and more typically, 50-500 mg. In certain embodiments, the composition includes a dosage of the compound and a volume of an aqueous solvent, such as water or saline, equal to 100% of the dosage unit minus the dose of agent. In certain embodiments, the ratio of active agent to carrier is between 1:10000 and 1:1 or from between 1:5000 and 1:1, or from between 1:1000 to between 1:1.

Ideally the compounds should be administered to achieve peak plasma concentrations of the active compound of from about 0.2 to 70 μM, preferably about 1.0 to 10 μM. This may be achieved, for example, by the intravenous injection of an appropriate concentration of the active ingredient, optionally in saline, or administered as a bolus of the active ingredient.

The concentration of the compounds in the drug composition will depend on absorption, inactivation and excretion rates of the extract as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The compounds may be administered at once, or may be divided into a number of smaller doses to be administered at varying intervals of time.

A typical mode of administration of the compound is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compounds can be administered as a component of an elixir, suspension, syrup, wafer, chewing gum or the like. A syrup may contain, in addition to the active compounds, sucrose as a sweetening agent and certain preservatives, dyes and colorings and flavors. The compounds can also be mixed with other active materials that do not impair the desired action, or with materials that supplement the desired action, such as antibiotics, antifungals, anti-inflammatories, or other anti-autoimmune compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In another embodiment, the compounds are prepared with carriers that will protect them against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art.

Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

### VIII. SYNTHESIS OF THE ACTIVE COMPOUNDS

### Formylation of a Substituted Phenol:

The starting material for this process is a substituted phenol (A), which can be purchased or can be prepared by any known means to those of ordinary skill in the art. In one embodiment, formylation of the compound of formula (A) results in the formation of an aldehyde of formula (B). The said substituted phenol can be coupled with a paraformaldehyde in a compatible solvent at a suitable temperature with the appropriate coupling reagent to yield the corresponding aldehyde. Possible coupling reagents are any reagents that promote coupling, including but not limited to SnCl₄, BF₃, AlCl₃, FeI₃, or ZnCl₂, preferably SnCl₄.

The formylation reaction can be carried out at any temperature that achieves the desired result, i.e., that is suitable for the reaction to proceed at an acceptable rate without promoting decomposition or excessive side products. The preferred temperature is room temperature.

Any reaction solvent can be selected that can achieve the necessary temperature, can solubilize the reaction components and inert to the reagents. Nonlimiting examples are any aprotic solvent including, but not limited to the alkyl solvents, such as hexane and cyclohexane, toluene, acetone, ethyl acetate, dithianes, triethylamine (TEA), tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably TEA.

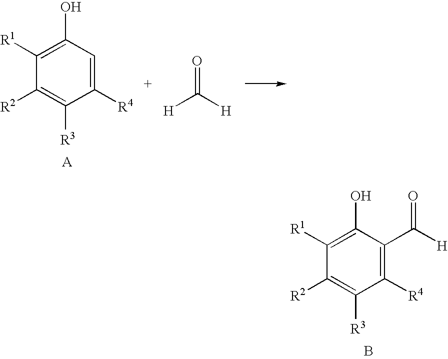

wherein R¹, R³, and R⁴ are selected independently from the groups that include hydrogen, alkyl, alkenyl, alkynyl, cycloalkyl, cycloalkenyl, cycloalkynyl, aryl, alkaryl, arylalkyl, heterocyclic, heteroaryl, sulfonyl, sulfanyl, sulfinyl, sulfamoyl, carboxylic acid, amide, nitro, cyano, azide, phosphonyl, phosphinyl, phosphoryl, phosphine, carbamate, ester, alkcarbonyl, carbonyl, a residue of a natural or synthetic amino acid, or carbohydrate or XR⁷ (X═O, NR⁸ or S).

### Reduction of Aldehyde:

In another embodiment of the present invention, reducing the compound of formula B results in the formation of the alcohol of formula C using a reducing agent such as NaBH₄. The reduction reaction can be carried out at any temperature that achieves the desired result, i.e., that is suitable for the reaction to proceed at an acceptable rate without promoting decomposition or excessive side products. The preferred temperature is room temperature.

Any reaction solvent can be selected that can achieve the necessary temperature, can solubilize the reaction components and inert to the reagents. Nonlimiting examples are any aprotic solvent including, but not limited to the alkyl solvents, such as hexane and cyclohexane, toluene, acetone, ethyl acetate, dithianes, triethylamine (TEA), tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably TEA.

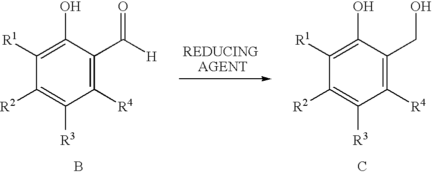

wherein R¹, R³, and R⁴ are selected independently from the groups that include hydrogen, alkyl, alkenyl, alkynyl, cycloalkyl, cycloalkenyl, cycloalkynyl, aryl, alkaryl, arylalkyl, heterocyclic, heteroaryl, sulfonyl, sulfanyl, sulfinyl, sulfamoyl, carboxylic acid, amide, nitro, cyano, azide, phosphonyl, phosphinyl, phosphoryl, phosphine, carbamate, ester, alkcarbonyl, carbonyl, a residue of a natural or synthetic amino acid, or carbohydrate or XR⁷ (X═O, NR⁸ or S).

### Coupling of a Substituted Phenol:

Alternatively, the substituted phenol can be formed using a ketone. Again, the starting material for this process is a substituted phenol (A), which can be purchased or can be prepared by any known means to those of ordinary skill in the art. In one embodiment, the compound of formula (A), is optionally protected, with an appropriate protecting group, as taught by Greene et al. Protective Groups in Organic Synthesis, John Wiley and Sons, Second Edition, 1991. Coupling of this optionally protected alcohol with an appropriate ketone results in the direct formation of an alcohol of formula (C′). The said substituted phenol can be coupled with the ketone in a compatible solvent at a suitable temperature with the appropriate base to yield the corresponding aldehyde. Possible coupling reagents are any reagents that promote coupling, including but not limited to lithiates, including, BuLi.

The formylation reaction can be carried out at any temperature that achieves the desired result, i.e., that is suitable for the reaction to proceed at an acceptable rate without promoting decomposition or excessive side products. The preferred temperature is room temperature.

Any reaction solvent can be selected that can achieve the necessary temperature, can solubilize the reaction components and inert to the reagents. Nonlimiting examples are any aprotic solvent including, but not limited to the alkyl solvents, such as hexane and cyclohexane, toluene, acetone, ethyl acetate, dithianes, triethylamine (TEA), tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably TEA.

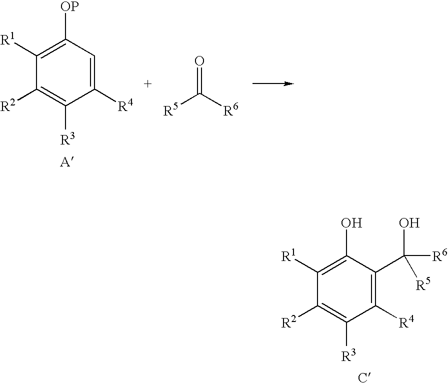

### Formation of Epoxides:

In yet a further embodiment of the present invention, the epoxidation of the compounds of formula (C) or (C′) yields compound (D). In another embodiment of the present invention, the compound of formula (D) is subjected to further oxidation resulting in the compound of formula (E) and (F). The formation of the monoepoxide (D) results from oxidizing the alcohol of formula (C) with an oxidizing agent such as sodium periodate (NaIO₄). Upon further oxidation of the monoepoxide (D) using oxidizing agents such as mCPBA give rise to the compound of formula (E) and (F). The oxidation reaction can be carried out at any temperature that achieves the desired result, i.e., that is suitable for the reaction to proceed at an acceptable rate without promoting decomposition or excessive side products. The preferred temperature is room temperature.

Any reaction solvent can be selected that can achieve the necessary temperature, can solubilize the reaction components and inert to the reagents. Nonlimiting examples are any aprotic solvent including, but not limited to the alkyl solvents, such as hexane and cyclohexane, toluene, acetone, ethyl acetate, dithianes, triethylamine (TEA), tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably TEA.

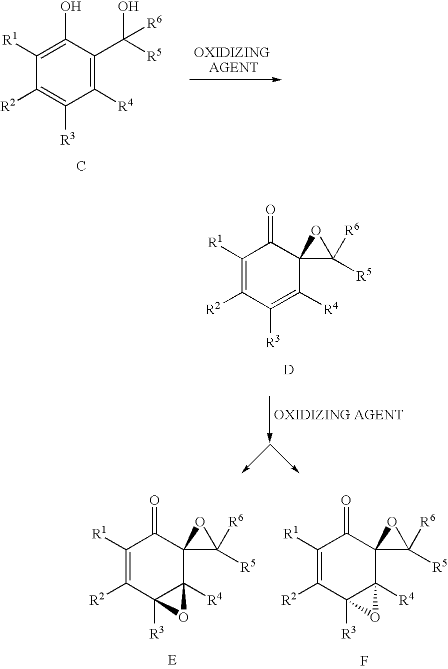

It is yet a further embodiment of the present in invention to further oxidize the compounds of formula (E) or (F) to give the stereoselective compounds of formula (G) or (H) respectively. Treating the compounds of formula (E) or (F) with an oxidizing agent such as hydrogen peroxide/NaOH yield the triepoxide (G) or (H) respectively. The oxidation reaction can be carried out at any temperature that achieves the desired result, i.e., that is suitable for the reaction to proceed at an acceptable rate without promoting decomposition or excessive side products. The preferred temperature is room temperature.

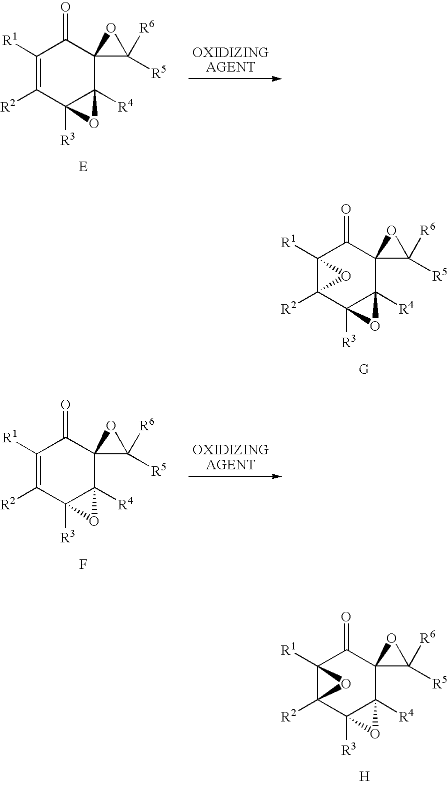

It is also a further embodiment of the present invention to provide for the compound of formula (J). The monoepoxide compound of formula (D) can be further oxidized to give the diepoxide compound of formula (J) using oxidizing agents such as hydrogen peroxide/NaOH. The oxidation reaction can be carried out at any temperature that achieves the desired result, i.e., that is suitable for the reaction to proceed at an acceptable rate without promoting decomposition or excessive side products. The preferred temperature is room temperature.

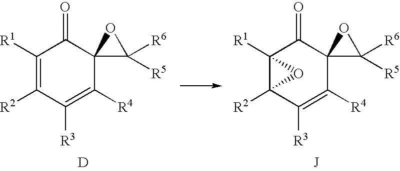

In another embodiment of the invention, the sulfur analogs are desired. Therefore, the sulfur analogs corresponding to the compounds of the invention can be prepared following the same foregoing general methods, beginning with the corresponding sulfur containing starting material.

### Formation of Cyclopropyls:

In yet a further embodiment of the present invention, the cyclopropanation of the compounds of formula (C) or (C′) yields compound (K). The formation of compound (K) results from eliminating the alcohol of formula (C) with an acid to form the alkene. The alkene can be reacted with the appropriate carbene to form the cyclopropane (K). The appropriate carbene can be made by any means known in the art. In particular, the carbene can be made via a-elimination. For example, dichlorocarbene can be made by treatment of chloroform with a base. Alternatively, the carbene can be made via the Simmons-Smith procedure with Zn—Cu, or Zn and Cu—X (wherein X is a halide), and in particular Zn and Cu—X in the presence of TiX₄. The carbene also can be made by the disintegration of certain types of double bonds, such as the photolysis of a ketene, the isoelectronic decomposition of diazoalkanes, and the decomposition of diazirines (which are isometric with diazoalkanes). Alternatively, ylides, such as R₂P≡CR⁷, R₂S(O)—CR⁷R⁸, such as Trost's and Corey's sulfur ylides, or R(NR₂)S(O)—CR⁷R⁸, that mimic a carbene, or transition metal-carbene complexes, such as Lₙ,M=CR⁷R⁸, wherein M is a metal and L is a ligand, and in particular, when M is Fe, may also be used to form the desired cyclopropane.

Regardless, the coupling reaction with the carbene can be carried out at any temperature that achieves the desired result, i.e., that is suitable for the reaction to proceed at an acceptable rate without promoting decomposition or excessive side products. The preferred temperature is room temperature. In the same way, any of the carbon-carbon pi bonds in compound (C) or (C′) can be reacted with the appropriate carbene to form the desired cyclopropane.

Any reaction solvent can be selected that can achieve the necessary temperature, can solubilize the reaction components and inert to the reagents. Nonlimiting examples are any aprotic solvent including, but not limited to the alkyl solvents, such as hexane and cyclohexane, toluene, acetone, ethyl acetate, dithianes, triethylamine (TEA), tetrahydrofuran (THF), dioxane, acetonitrile, dichloromethane, dichloroethane, diethyl ether, pyridine, dimethylformamide (DMF), dimethylsulfoxide (DMSO), dimethylacetamide or any combination thereof, though preferably TEA.

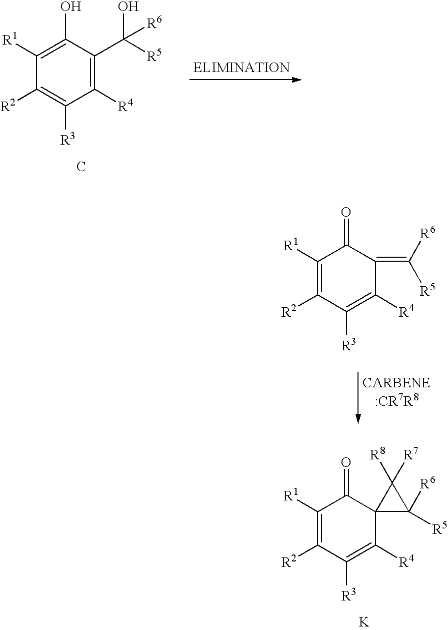

### Hydroxylation of Double Bond:

The hydroxylation of double bonds is a well known reaction in organic chemistry. For example hydroxylation of an olefin bond may be accomplished by hydroboration followed by oxidative treatment of the intermediate borane to provide the alcohol (see Brown et al, J. Am. Chem. Soc.; 1964; 86(17) pp 3565-3566). Many other methods are also suitable.

### Dihydroxylation of Double Bonds:

Many reagents are capable of dihydroxylating a double bond (see Sheldon and Koci, “Metal-Catalyzed Oxidations of Organic Compounds”, pp. 162-171, 294-296, Academix Press, New York, 1981; Gunstone, Adv. Org. Chem. 1960, 1, 103-147). In one non-limiting example, dihydroxylation of a double bond from the same face of the six-membered ring is achieved by treating a solution of an unsaturated compound with osmium tetraxoide followed by treatment with sodium sulfite to reduce the cyclic osmium addition intermediate (see Schroder, Chem. Rev., 1980, 80, 187-213).

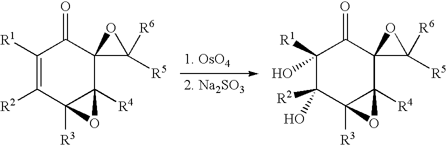

Enantioselective dihydroxylation reactions are also known in the art and can be accomplished to form specific stereoisomers of the diols (see Jacobsen, E. N.; Marko, I.; Mungall, W. S.; Schroeder, G.; Sharpless, K. B. J. Am. Chem. Soc. 1988, 110, 1968).

There are also many different known methods of forming trans-diols from olefins. For example a trans-diol may be formed from the di-epoxide by a Prevost oxidation reaction of the olefin (shown below).

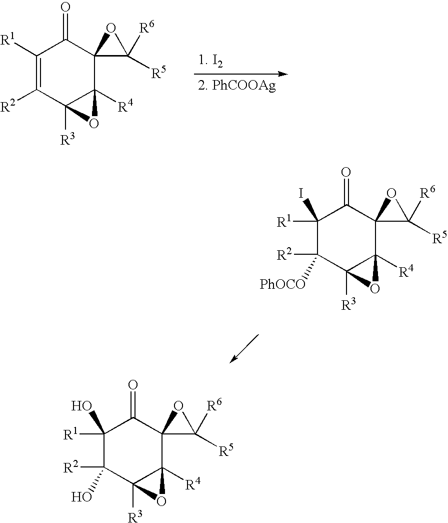

### Aminohydroxylation

1,2-aminoalcohols may be prepared by a variety of methods known in the art. For example, treatment of an epoxide with ammonia results in opening of the of the epoxide ring to form a 1,2-trans amino alcohol. Similar to the Sharpless asymmetric dihydroxylation discussed above, Sharpless and co-workers have reported the asymmetric amino hydroxylation of olefin bonds using with alkyl imido osmium compounds to form vicinal amino-alcohols (see McLeod et al. J. Chem. Soc., Perkin Trans. 1, 2002, 2733-2746).

### Conjugate Addition

In addition to hydroxylation of double bonds on the cyclohexenone compounds, conjugate addition of the α,β-enone group with a nucleophilic group may provide compounds with various substituents at the carbon atom beta to the carbonyl. Any nucleophilic group known in the art to add in a conjugate fashion to an enone may be used. Non-limiting examples include, but are not limited to, heteroatom nucleophiles and carbon nucleophiles, such as cuprate reagents and the like.

Various other synthetic reactions to derivatize double bonds are known in the art and may be used to prepare the compounds of the invention. Non-limiting examples include diamination of double bonds to provide vicinal diamines (Gomez et al, Synthesis, 1974, 504), azasulfenylation of double bonds (Trost et al., J. Am. Chem. Soc 1982, 104, 3225), 1,3-dipolar addition of double bonds to provide triazolines (see Padwa, “1,3-Dipolar Cycloaddition Chemistry,” Wiley, New York, 1934) Aminochlorination of enone bonds is known and may be used to prepare the compounds of the invention (see Chen et al., Eur. J. Org. Chem., 2004, 3097-3101). Synthesis of Substituted Benzyl Alcohols.

To a magnetically stirred solution of the phenol (20 mmol) in toluene (10 mL), triethylamine (8 mmol) was added followed by SnCl₄ (2 mmol). After stirring for 0.5 h at room temperature, paraformaldehyde (40 mmol) was added and the slurry heated to 95° C. for 16 h. The reaction mixture was cooled and poured into water (40 mL) and acidified to pH 2 with 1 N HCl. The aqueous layer was extracted with diethyl ether (3×60 mL). The combined organics were washed with brine, dried (sodium sulphate) and concentrated under vacuo. The crude product (B) was subjected to reduction with NaBH₄ (1.5 equivalents) in MeOH at 0° C. After stirring the reaction for 1 h, the reaction mixture was quenched with a saturated solution of ammonium chloride and acidified to pH 4 with 1 N HCl. MeOH was removed under vacuum and the aqueous layer extracted with ethyl acetate (2×50 mL). The combined organics were washed with brine, dried (sodium sulfate) and concentrated under vacuo. Flash chromatography using 1:4::ethyl acetate:hexanes yielded the desired product C in moderate yield over 2 steps (35-60%).

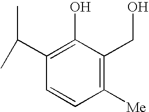

¹H NMR (CDCl₃): 7.9 (br s,1H), 7.06 (d, 1H, J=7.8 Hz), 6.7 (d, 1H, J=7.8 Hz), 4.94 (s, 2H), 3.3 (m, 1H), 2.24 (s, 3H), 1.24 (d, 6H, J=6.9 Hz).

¹³C NMR (CDCl₃): 154.02, 133.90, 132.89, 125.62, 122.19, 121.90, 61.38, 26.75, 22.98, 19.40.

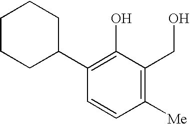

¹H NMR (CDCl₃): 7.92 (br s, 1H), 7.04 (d, 1H, J=7.8 Hz), 4.93 (s, 2H), 2.9 (m, 1H), 2.23 (s, 3H), 1.8 (m, 6H), 1.36 (m, 4H).

¹³C NMR (CDCl₃): 153.91, 133.11, 132.85, 126.12, 122.20, 121.90, 61.33, 36.88, 33.5, 27.35, 26.69, 19.39.

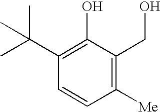

¹H NMR (CDCl₃): 8.1 (br s, 1H), 7.15 (d, 1H, J=7.8 Hz), 6.67 (d, 1H, J=7.8 Hz), 4.92 (s, 2H), 2.25 (s, 3H), 1.43 (s, 9H).

¹³C NMR (CDCl₃): 156.17, 135.36, 133.54, 126.43, 123.06, 121.55, 61.26, 34.69, 29.84, 19.26.

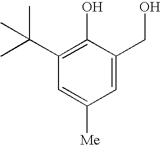

¹H NMR (CDCl₃): 7.48 (s, 1H), 7.02 (s, 1H), 6.68 (s, 1H), 4.78 (s, 2H), 2.22 (s, 3H), 1.38 (s, 9H).

¹³C NMR (CDCl₃): 153.15, 137.19, 128.41, 127.79, 126.49, 124.80, 65.20, 34.76, 29.81, 20.85

### Example 1

### Formation of Monoepoxide Using Sodium Periodate

To a magnetically stirred solution of C (2 mmol) in MeOH (12 mL), a solution of NaIO₄ (2.2 mmol) in 3 mL water was added dropwise at 0° C. After one minute of stirring a precipitate began to appear. After stirring for another 20 minutes the precipitate was filtered and washed with CHCl₃. Water was added and the aqueous layer was extracted with chloroform. The combined organics were washed with brine, dried (sodium sulfate) and concentrated under vacuo. Purification by flash chromatography using 1:7::ethyl acetate:hexanes yielded the desired product (D, 80%).

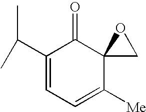

¹H NMR (CDCl₃): 6.87 (d, 1H, J=6.6 Hz), 6.3 (m, 1H), 3.22 (d, 1H, J=8.1 Hz), 3.15 (d, 1H, J=8.1 Hz), 2.9 (m, 1H), 1.79 (d, 3H, J=1.5 Hz), 1.08 (d, 3H, J=2.1 Hz), 1.05 (d, 3H, J=2.1 Hz).

¹³C NMR (CDCl₃): 195.31, 144.45, 141.75, 135.62, 124.02, 59.27, 58.81, 26.45, 22.14, 21.88, 16.42.

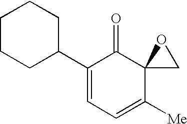

¹H NMR (CDCl₃): 6.8 (m, 1H), 6.23 (m, 1H), 3.14 (m, 1H), 3.05 (m, 1H), 2.52 (m, 1H), 1.65 (m, 8H), 1.27 (m, 2H), 1.1 (m, 3H)

¹³C NMR (CDCl₃): 195.41, 144.30, 140.89, 136.13, 124.10, 59.12, 58.70, 35.79, 32.56, 32.31, 26.68, 26.59, 26.28, 16.15.

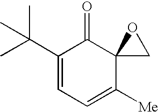

¹H NMR (CDCl₃): 6.94 (d, 1H, J=6.3 Hz), 6.27 (d, 1H, J=6.3 Hz), 3.14 (AB quartet, 2H, J=8.1 Hz), 1.79 (s, 3H), 1.21 (s, 9H).

¹³C NMR (CDCl₃): 195.07, 145.18, 143.06, 136.44, 123.98, 59.85, 58.44, 34.34, 29.14, 16.19.

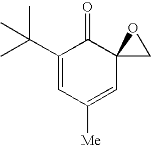

¹H NMR (CDCl₃): 6.79 (d, 1H, J=2.1 Hz), 5.68 (d, 1H, J=2.1 Hz), 3.23 (d, 1H, J=8.1 Hz), 3.01 (d, 1H, J=8.1 Hz), 1.98 (s, 3H), 1.20 (s, 9H).

¹³C NMR (CDCl₃): 194.42, 144.74, 140.25, 135.79, 130.94, 58.92, 57.26, 34.55, 29.23, 22.00.

### Example 2

### Formation of Diepoxides from the Monoepoxides Using mCPBA

To a magnetically stirred solution of D (1 mmol) in methylene chloride (10 mL), mCPBA was added and the reaction mixture stirred for 14 h. The mixture was diluted with methylene chloride and washed twice with saturated sodium carbonate. The combined organics were washed with brine, dried (sodium sulfate) and concentrated under vacuo. Purification by flash chromatography using 1:7::ethyl acetate:hexanes yielded 2 products that were separated. Stereochemical assignments were made by comparison with literature data. Typically the higher R_{F} spot was assigned the stereochemistry F and the lower R_{F} spot was assigned the stereochemistry E. The combined yield for the reaction was typically 70%.

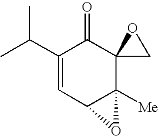

¹H NMR (CDCl₃): 6.95 (d, 1H, J=4.5 Hz), 3.46 (d, 1H, J=4.5 Hz), 3.42 (d, 1H, J=6.6 Hz), 3.1 (d, 1H, J=6.6 Hz), 2.85 (m, 1H), 1.31 (s, 3H), 1.04 (d, 3H, J=5.1 Hz), 1.02 (d, 3H, J=5.1 Hz).

¹³C NMR (CDCl₃): 190.71, 149.44, 136.89, 61.39, 55.66, 54.43, 50.11, 27.40, 21.67, 21.64, 16.16.

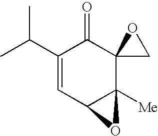

¹H NMR (CDCl₃): 6.9 (d, 1H, J=4.5 Hz), 3.51 (d, 1H, J=4.8 Hz), 2.96 (d, 1H, J=6.3 Hz), 2.87 (d, 1H, J=6.3 Hz), 2.79 (m, 1H), 1.32 (s, 3H), 1.06 (d, 3H, J=6.9 Hz), 0.97 (d, 3H, J=6.9 Hz).

¹³C NMR (CDCl₃): 191.06, 150.00, 136.40, 59.62, 57.92, 54.55, 52.66, 27.51, 21.69, 21.55, 15.63.

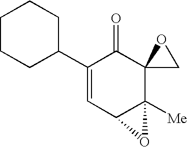

¹H NMR (CDCl₃): 6.89 (d, 1H, J=3.3 Hz), 3.44 (d, 1H, J=3.3 Hz), 3.38 (d, 1H, J=4.8 Hz), 3.09 (d, 1H, J=4.8 Hz), 2.49 (m, 1H), 1.71 (m, 5H), 1.3 (s, 3H), 1.33-1.03 (m, 5H).

¹³C NMR (CDCl₃): 190.89, 148.72, 137.42, 61.3, 55.59, 54.42, 50.04, 36.85, 32.25, 26.58, 26.56, 26.26, 16.02.

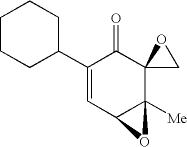

¹H NMR (CDCl₃): 6.83 (d, 1H, J=3.6 Hz), 3.5 (d, 1H, J=3.6 Hz), 2.94 (m, 1H), 2.82 (m, 1H), 2.45 (br t, 1H), 1.73-1.52 (m, 5H), 1.13 (s, 3H), 1.26-0.96 (m, 5H)

¹³C NMR (CDCl₃): 191.27, 149.21, 136.87, 59.58, 57.76, 54.52, 52.51, 36.85, 32.31, 31.99, 26.50, 26.41, 26.14, 15.39.

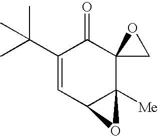

¹H NMR (CDCl₃): 6.92 (d, 1H, J=3.3 Hz), 3.49 (d, 1H, J=3.3 Hz), 2.89 (d, 1H, J=4.2 Hz), 2.79 (d, 1H, J=4.2 Hz), 1.31 (s, 3H), 1.14 (s, 9H).

¹³C NMR (CDCl₃): 191.21, 151.52, 136.77, 60.76, 57.35, 54.44, 51.60, 35.05, 28.90, 14.97.

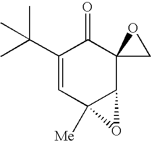

¹H NMR (CDCl₃): 6.78 (s, 1H), 3.44 (d, 1H, J=6.6 Hz), 3.21 (s, 1H), 3.03 (d, 1H, J=6.6 Hz), 1.57 (s, 3H), 1.12 (s, 9H).

¹³C NMR (CDCl₃): 190.07, 149.46, 142.55, 63.23, 54.31, 54.00, 51.34, 35.16, 29.05, 21.07.

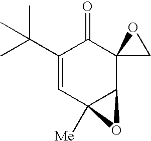

¹H NMR (CDCl₃): 6.72 (s, 1H), 3.13 (s, 1H), 2.89 (s, 2H), 1.57 (s, 3H), 1.09 (s, 9H).

¹³C NMR (CDCl₃): 190.07, 150.01, 142.08, 60.59, 58.08, 54.85, 52.96, 35.03, 28.98, 21.11.

### Example 3

### Oxidation of Monoepoxides to Diepoxides Using Hydrogen Peroxide

To a magnetically stirred solution of the monoepoxide (1 mmol) in MeOH (10 mL) at room temperature, 1 N NaOH (0.47 mL) was added followed immediately by the addition of 30% H₂O₂ (1.5 mmol). After 40 minutes of stirring at room temperature, water was added (40 mL) and the aqueous layer extracted with ethyl acetate (3×60 mL). The combined organics were washed with brine, dried (sodium sulfate) and concentrated under vacuo. Flash chromatography using 1;6::ethyl acetate:hexanes yielded the desired product in moderate yield (60%).

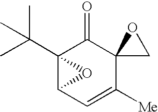

¹H NMR (CDCl₃): 6.08 (m, 1H), 3.73 (d, 1H, J=3 Hz), 2.94 (s, 2H), 1.61 (s, 3H), 1.09 (s, 9H).

¹³C NMR (CDCl₃): 200.05, 141.71, 123.21, 64.42, 60.21, 56.4, 52.92, 32.10, 25.76, 15.67.

### Example 4

### Dihydroxylation of Epoxide Compounds

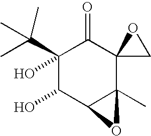

The t-butyl substituted cis-diepoxide enone (2.0000 g, 9 6 mmol) was dissolved in 2 ml of a 1:1 solution of water and acetone. This solution was stirred and then chilled to 0° C. in an ice/water bath. Once cooled osmium tetraoxide (122 mg, 0.48 mmol, 0.05 equiv.) was added. Finally, 5.6 ml of a 50% solution 4-methylmorpholine oxide in water was added (2.8 g, 24 mmol, 2.5 equiv.). The reaction mixture was allowed to stir over night warming to room temperature. The reaction mixture was transferred to a separatory funnel and extracted with ethyl acetate (3×30 ml). The ethyl acetate extracts were pooled, washed with brine, dried with MgSO₄, and filtered. Ethyl acetate was removed under reduced pressure resulting in a thick oil. The product was purified on silica eluting with 1:1 hexanes/ethyl acetate. The product was then crystallized from hot hexanes and ethyl acetate giving white crystals (73% yield).

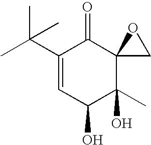

The t-butyl substituted monoepoxide dienone (1.0000 g, 5.2 mmol) was dissolved in 20 ml of a 1:1 solution of water and acetone. The solution was stirred and cooled to 0° C. in an ice/water bath. Once cooled osmium tetraoxide (16 ng, 0.26 mmol, 0.05 equiv.) was added. Finally, 1.34 ml of a 50% solution of 4-methylmorpholine oxide in water was added (0.6703 g, 5.7 mmol, 1.1 equiv.). The reaction mixture was allowed to stir over night warming to room temperature. The reaction mixture was transferred to a separatory funnel and extracted with ethyl acetate (3×30 ml). The ethyl acetate extracts were pooled, washed with brine, dried with MgSO₄, and filtered. Ethyl acetate was removed under reduced pressure resulting in a thick oil. The product was purified on silica eluting with 1:1 hexanes/ethyl acetate. The diol product was then crystallized from hot hexanes and ethyl acetate giving white crystals (45% yield).

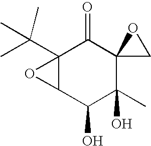

To a stirring solution of the dihydroxylated monoepoxide compound (0.1000 g, 0.44 mmol) of methanol was added 0.21 ml of a 1 M NaOH solution followed immediately by 0.08 ml of a 30% hydrogen peroxide in water solution (0.0226 g, 0.66 mmol, 1.5 equiv.). The reaction was allowed to stir at room temperature for 1 hour. After 1 hour, the reaction mixture was transferred to a separatory funnel and extracted with ethyl acetate (3×20 ml). The ethyl acetate extracts were pooled, washed with brine, dried with magnesium sulfate, and filtered. The solvent was removed under reduced pressure. The resulting oil was purified on silica eluting with 1:1 hexanes/ethyl acetate. After removal of the solvents under reduced pressure the solid was crystallized from hot hexanes and ethyl acetate giving a white solid (75% yield).

The invention has been described with reference to its preferred embodiments. Variations and modifications of the invention will be obvious to those skilled in the art from the foregoing description. It is intended that all of these variations and modifications be included within the scope of the appended claims.

What is claimed is:

1. A compound of the formula (I) or (II): 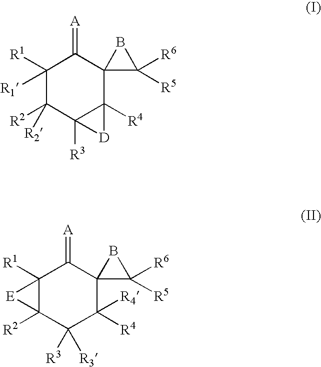
or a pharmaceutically acceptable salt. ester, or prodrug thereof, wherein:
A, B, D and E are independently O, S, NR⁷ or CR⁷R⁸;
R¹, R², R³, R⁴, R⁵ and R⁶ are independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, carbonyl, carboxylic acid, ester, carbamate, amide, amine, hydroxyl, alkoxy, nitro, cyano, azide, sulfonyl, sulfanyl, sulfinyl, sulfamoyl, phosphonyl, phosphinyl, phosphoryl, phosphine, a residue of a natural or synthetic amino acid, a residue of a natural or synthetic carbohydrate or XR⁹ (wherein X═O, S or NR¹⁰);
alternatively, one or more of R¹ or R^{1′} and R² or R^{2′}, R² or R^{2′} and R³ or R^{3′}, R³ or R^{3′} and R⁴ or R^{4′}, R⁴ or R^{4′} and R⁵, or R⁵ and R⁶, come together to form a bridged compound, preferably as a 3, 5, 6 or 7 membered ring, to form a cycloalkyl, cycloalkenyl, cycloalkynyl, aryl, heterocyclic, or heteroaryl;
each R⁷, R⁸, R⁹ and R¹⁰ is independently hydrogen, alkyl, alkenyl, alkynyl, cycloalkyl, cycloalkenyl, cycloalkynyl, aryl, alkaryl, arylalkyl, heterocyclic, heteroaryl, alkcarbonyl, a residue of a natural or synthetic amino acid or a residue of a natural or synthetic carbohydrate;
R^{1′}, R^{2′}, R^{3′} and R^{4′} are independently hydrogen, halogen, azide, —OH, —OR⁹, —NH₂, —NHR⁹, —N(R⁹)₂, —SH, —SR⁹, —OC(O)R⁹, —OC(O)OR⁹, —OC(O)N(R9)2₂, —NR⁹C(O)R⁹, —NR⁹C(O)OR⁹, —NR⁹C(O)N(R⁹)₂, —NR⁹SO₂R⁹, —SO₂N(R⁹)₂, —S(O)R⁹, —S(O)₂R⁹, —N—OR⁹, a residue of a natural or synthetic amino acid, or a residue of a natural or synthetic carbohydrate;
wherein two R⁹ groups on the same nitrogen may form an optionally substituted 3-8 membered heterocyclic or heteroaryl ring;
with the provisos that in Formula I, when R¹ or R^{1′} are hydrogen, then R² and R^{2′} are not hydrogen and when R² or R^{2′} are hydrogen, then R¹ and R^{1′} are not hydrogen; and in formula II, when R³ or R^{3′} are hydrogen, then R⁴ and R^{4′} are not hydrogen and when R⁴ or R^{4′} are hydrogen, then R³ and R^{3′} are not hydrogen.

2. The compound of claim 1, wherein the compound has the formula (I).

3. The compound of claim 1, wherein the compound has the formula (II).

4. The compound of claim 1, wherein A, B, D and E are O.

5. The compound of claim 1, wherein R^{1′}, R^{2′}, R^{3′} and R^{4′} are independently —OH, —OR⁹, —NH₂, —NHR⁹, or —N(R⁹)₂.

6. The compound of claim 1, wherein R¹, R², R⁴, R⁵ and R⁶ are independently hydrogen or alkyl.

7. The compound of claim 1, wherein R⁵ and R⁶ are hydrogen.

8. The compound of claim 1, wherein R³ is hydrogen.

9. The compound of claim 1, wherein R⁴ is alkyl.

10. The compound of claim 1, wherein:
R¹ and R² are independently hydrogen or alkyl;
R⁴ is hydrogen or alkyl;
R⁵ and R⁶ are hydrogen; and
A, B, D and E are O.

11. The compound of claim 10, wherein the compound has the formula (I), and R^{1′} and R^{2′} are independently OH or NH₂.

12. The compound of claim 10, wherein the compound has the formula (II), and R^{3′} and R^{4′} are independently OH or NH₂.

13. The compound of claim 11, wherein R¹ is cyclohexyl, isopropyl or tert-butyl.

14. The compound of claim 1, wherein the compound has the structure

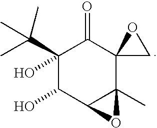

15. The compound of claim 1, wherein the compound has the structure

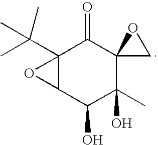

16. A pharmaceutical composition for the treatment or prophylaxis of an autoimmune, inflammatory or proliferative disorder comprising an effective treatment amount of the compound of claim 1 in combination with a pharmaceutically acceptable carrier.

17. A method for the treatment of an autoimmune or inflammatory disease in a patient comprising administering to the patient with an autoimmune or inflammatory disease an effective amount of a compound of claim 1, optionally with a pharmaceutically acceptable carrier.

18. A method for the treatment of abnormal cell proliferation in a patient comprising administering to the patient with an autoimmune or inflammatory disease an effective amount of a compound of claim 1, optionally with a pharmaceutically acceptable carrier.

19. The method of claim 17, wherein the abnormal cell proliferation is cancer.

20. A pharmaceutical composition for the treatment or prophylaxis of an autoimmune, an inflammatory or a proliferative disorder comprising an effective treatment amount of the compound of claim 1 in combination with another active agent and a pharmaceutically acceptable carrier.

21. A method for the treatment or prophylaxis of an autoimmune or inflammatory disease in a patient comprising administering to the patient with an autoimmune or inflammatory disease an effective amount of a compound of claim 1 in combination with one or more other active agent(s), optionally with a pharmaceutically acceptable carrier.

22. A method for the treatment or prophylaxis of abnormal cell proliferation in a patient comprising administering to the patient with a proliferative disorder an effective amount of a compound of claim 1 in combination with one or more other active agent(s), optionally with a pharmaceutically acceptable carrier.
